SEQUENCE LISTING (16 sequences)

SEQ ID NO: 1 - DNA, 4806 bp, Erythroparvovirus
gaagttgaccgcgaagctccacagcatgtttgcggaatgcggggcaggcgaggagaccaagtactattacagcgtgccgcgtgaccggcacgggaaagtacttaggtgtgacgaggagttcatccgcacttatttgttgaaaaaggtcccgctagacggtgtgtggtgggcttggacgactattgacaaatttaaacccatagttctcagcgagacccggcgccgctccctggcagccgctgagcggctgaacgacggtgggacagaccaggcgctagtcccggtccaacctccgccggccaagcggacgcgggcttctgacgaatttgtgactttatgcaactggcttgcccaggaaggcattctgaccgaacaaaagtggagagcgactgatcttaacgggtatgtacgacagtatgcctctatgcacggccgtcagcaggtagcggccgcgctgatgatggcccgtaacataatccttgataccatggccaacagccattttctcaccagaggccccccagccgtggtagccaccccggcgtgggaggaaaacagggtggtccaattgttgcgccttcagggctatgatccgcgggtggtggggcagtatattctagcttgggcggacggaatgacggggaagaggaatacactttggctctatgggcctcccagcacgggaaaaacaaatctcgccaatgcgatctgtcagtgcgtgcccgtgtacgggatggttaattggaccaatgaaaattttccatttaatgatgcggcagataaatgtctgattttgtgggatgagggacgcatcagtagtaaaattgtggaggcctgtaaggccatcttgggcggcatgccggttcgagtagaccaaaaatgtaaggggagcaccgccctccggccgaccccggtgttggttaccagcaacggggatatgaccatcgtcagagatggcaataccagtactttgatccatcggcctgccctgaaagatcgcatggtgaggctagacttcaatgtaaagttgcccgctgaatatgggttgttgacccccagtgatgtgcgggcctggttggcaggttgtcgggaccaccccccactggtcgatgcgatcaacatggggtttacttttcctgtgggcagtgatgctcacaaggaggacgagggcgatgacgacgccgaccatagcggcagtcgtctccacagcagtcccttccagtcacgtcgcgtcccggacacccccagtgacggaatctggcccgaggacgacgagtcagactctgacgcggagctctccgagggcgacctcaacgacatcctccccctcgtcaactcctgggaaagttccgtgtccccgacataccggctcgaacgacccatttgtggtgtcgattacgtgtggtcgacccttctcaacggggctgtgtgttcggcggaacacgccacggcggcgaagccgcctccttgcttcgactgctatcgggtgcactccatccttgggtgttgggcggttcgggaccgcgaaaacgggcccgaaaccggcgacctaaggcgttgtgttctacaccgtaacagtaaagatcccctggatgttcttccctgtcaccgctgtacggcactgagtggcttagaacactttgtgtctagcgactctcccccaaaagtgtacggggccgccacggaggtggtggcctttggcgccggtggtcaggaaaccccgaaacaaacgccaactacccagccccctgtggtgactccccctgccccacgccgtccacccgtggttaaagtgcttcccttgtccccccccgctcctccgcctccttcgccggctggcccgagcccttcgccccccccactcaacgtccgtcgtcgtttgagtttctgatgtcatgggtgaccatcctctgttcaattgggctcgccagcttgctcggtttaggctttcgcgtcgtccgctttggggccgtgggttcgggggggggagacggcgtcgtgccgtggaacatgcacctgctttggctgatttgcttgctgggggtggggatgtggtccatttaatagatctcttgaaacagcccagtatcctgcagagtgaagctagctcatttctagaacatttggtgttacgcaaattgcatctagaacatgtaattgagcccaataaagaccttcaggcaatatacgatgtggtcacgggtgatgaattagttgacaaagctgtggagcagttaactttcagtgggcaggaccctgaaaaattgggccagaactacgcccaaaaacggcaacatgtccaggaacttttaaaagagtttgttggacatgtccgccggggggactatgacagtttaagaccggctaaccatgtagacgccccccctctgcaggacgtgggttcaccggacaccgcggatgtggaggaccctgatgaaccagaccctaaacgccctaaagtggagcccgggggacttggagaccctgttgagggaaacgggctggaccctcctccagatgttgatggaggcggtgtccacctacccggtgatcactaccttgggcctgggaatcctttacaaaatggccccccctctgacatggtggatgcgatcgcccgcgtccacgattaccggtacgcggctatcatggccatgggactcaacccgtacactcactataatatagccgatgccgaattgctcgaaaatcttaaaagccagactggggttcgggccgcggccgcccgagcctatttcgatctgaagaaaatgttattccctcatcttactttacaggccacactcccgggtgttccgagttgggcagcttcacaaacggaatcaaatatgcccggcgacgattctaacaccggaggtgtgggcggcggccaagctaacctcggcgtctgggcccagggggcgaccttcaccgatactgcggtgacctgttacatgacgaggcggtgttacctgccgtttaaccccgacccggtatatgaacccatcacacaccggggccgcaatgcgtataaagagggcccctaccagatggtcatgcgtcaccccgtatacggctacaagaccccctggcattaccctgatttaaacgccttaaacctatacttttccccagccgaatttcaatacttactagaacaatatgagtccattgtgccggcgagcatggacgtactcatcacagatatcattattaaggaagttagccaaatgaacaatacaacccaggtggctgacagcggcactggcgccatagccattctcaccgacgccgattataaatacccctacgtcctagggacggggcaagccaccttgcctccggacattcccacagacatgtatctactgccccaatatgcctatctcaccacagggctcattgagggcgaacaccacgaacaccatgtggccacggacaatcaagtggtcattcctaacagcgacacagatttctatgtgttagaacacagtcactttaccctacaccgcacaggtgacctattttcccacagttatcagtttccggaggccaaacccaaatcactggtgggggccagccaacatttcttcctgatggaaaaccccctctatgactcgaggttaaaagttctagaccgcactggcactaatgccaactggcgacacatcgccaaggaccactatgggtccaaaccccagaacttcttcccaggccccctcatcagttcacataccgcttatgcccatgatggtgttgccccacgatggggcgcgtgggcgtctgagatgcagtggctacagacgggtaccagtgtgggagatgaaccttatgctaggtggtcttttaggcccggccccgcctgccaacctttttcccacacaccagctgacaaccccacagaagaccaaccatctgtgggcgtagacgcggtctcttggggtctacacgaaaacaccagtgtggacacccccggcctgaaccgcgggcggctgcccgatgatcgggaacaggccaaacaactagggggattagatgaaaagttctggccggttacccgcctaaaccaggctcttaaagaagaccaggtttcccacgcacggcccctaatgcccggttcagtgtggaacgaacgggctctccactacgagagtcagatctgggccaaaattccccgctgtgataggagtttcatggctgacagccccatgatgggcggctggggtttcgccgcaccgccgccaatgattttcatcaaaatgttgcccaccccggcgcccctcggtacggacaccgtgaccgaaaatacaacagaaaaggctgggatacttcatcagtacgcagtgtttaaccttaccgtcaagctggtgtttaacttaaagaagcgcaccatgacgggccgctggaacccccagcccgccgtgtaccctccctctgctcggggacacatgccctatgttctgtacaccccccgcgacctcccgggtaatcaacaccctgacgtgtacgggcacgatggctatgagcggtccgaacactgttggaccgctaaagcgcgtgtcgcccacttgtaatgcaataaacctgatgtccaccagatggcagtgtctcgcgtcatttgtccgggaggggcccaaaagctactatgtcacctgatcgcggatggcgccgttccaagggcggacttacaagcgcgggaaattcgaaaagcgcgccggcgggaaatttgaaaaaagcgcgggaatttcaaaaaaagcgcgggaaagtccgttatattgaatccagccgaccgtgacccattgacaa

SEQ ID NO: 2 - DNA, 1620 bp, Erythroparvovirus
  CDS (1)..(1620)
atgacgaggcggtgttacctgccgtttaaccccgacccggtatatgaacccatcacacaccggggccgcaatgcgtataaagagggcccctaccagatggtcatgcgtcaccccgtatacggctacaagaccccctggcattaccctgatttaaacgccttaaacctatacttttccccagccgaatttcaatacttactagaacaatatgagtccattgtgccggcgagcatggacgtactcatcacagatatcattattaaggaagttagccaaatgaacaatacaacccaggtggctgacagcggcactggcgccatagccattctcaccgacgccgattataaatacccctacgtcctagggacggggcaagccaccttgcctccggacattcccacagacatgtatctactgccccaatatgcctatctcaccacagggctcattgagggcgaacaccacgaacaccatgtggccacggacaatcaagtggtcattcctaacagcgacacagatttctatgtgttagaacacagtcactttaccctacaccgcacaggtgacctattttcccacagttatcagtttccggaggccaaacccaaatcactggtgggggccagccaacatttcttcctgatggaaaaccccctctatgactcgaggttaaaagttctagaccgcactggcactaatgccaactggcgacacatcgccaaggaccactatgggtccaaaccccagaacttcttcccaggccccctcatcagttcacataccgcttatgcccatgatggtgttgccccacgatggggcgcgtgggcgtctgagatgcagtggctacagacgggtaccagtgtgggagatgaaccttatgctaggtggtcttttaggcccggccccgcctgccaacctttttcccacacaccagctgacaaccccacagaagaccaaccatctgtgggcgtagacgcggtctcttggggtctacacgaaaacaccagtgtggacacccccggcctgaaccgcgggcggctgcccgatgatcgggaacaggccaaacaactagggggattagatgaaaagttctggccggttacccgcctaaaccaggctcttaaagaagaccaggtttcccacgcacggcccctaatgcccggttcagtgtggaacgaacgggctctccactacgagagtcagatctgggccaaaattccccgctgtgataggagtttcatggctgacagccccatgatgggcggctggggtttcgccgcaccgccgccaatgattttcatcaaaatgttgcccaccccggcgcccctcggtacggacaccgtgaccgaaaatacaacagaaaaggctgggatacttcatcagtacgcagtgtttaaccttaccgtcaagctggtgtttaacttaaagaagcgcaccatgacgggccgctggaacccccagcccgccgtgtaccctccctctgctcggggacacatgccctatgttctgtacaccccccgcgacctcccgggtaatcaacaccctgacgtgtacgggcacgatggctatgagcggtccgaacactgttggaccgctaaagcgcgtgtcgcccacttgtaa

SEQ ID NO: 3 - protein, 539 aa, Erythroparvovirus
MTRRCYLPFNPDPVYEPITHRGRNAYKEGPYQMVMRHPVYGYKTPWHYPDLNALNLYFSPAEFQYLLEQYESIVPASMDVLITDIIIKEVSQMNNTTQVADSGTGAIAILTDADYKYPYVLGTGQATLPPDIPTDMYLLPQYAYLTTGLIEGEHHEHHVATDNQVVIPNSDTDFYVLEHSHFTLHRTGDLFSHSYQFPEAKPKSLVGASQHFFLMENPLYDSRLKVLDRTGTNANWRHIAKDHYGSKPQNFFPGPLISSHTAYAHDGVAPRWGAWASEMQWLQTGTSVGDEPYARWSFRPGPACQPFSHTPADNPTEDQPSVGVDAVSWGLHENTSVDTPGLNRGRLPDDREQAKQLGGLDEKFWPVTRLNQALKEDQVSHARPLMPGSVWNERALHYESQIWAKIPRCDRSFMADSPMMGGWGFAAPPPMIFIKMLPTPAPLGTDTVTENTTEKAGILHQYAVFNLTVKLVFNLKKRTMTGRWNPQPAVYPPSARGHMPYVLYTPRDLPGNQHPDVYGHDGYERSEHCWTAKARVAHL

SEQ ID NO: 4 - DNA, 2679 bp, Erythroparvovirus
atgggtgaccatcctctgttcaattgggctcgccagcttgctcggtttaggctttcgcgtcgtccgctttggggccgtgggttcgggggggggagacggcgtcgtgccgtggaacatgcacctgctttggctgatttgcttgctgggggtggggatgtggtccatttaatagatctcttgaaacagcccagtatcctgcagagtgaagctagctcatttctagaacatttggtgttacgcaaattgcatctagaacatgtaattgagcccaataaagaccttcaggcaatatacgatgtggtcacgggtgatgaattagttgacaaagctgtggagcagttaactttcagtgggcaggaccctgaaaaattgggccagaactacgcccaaaaacggcaacatgtccaggaacttttaaaagagtttgttggacatgtccgccggggggactatgacagtttaagaccggctaaccatgtagacgccccccctctgcaggacgtgggttcaccggacaccgcggatgtggaggaccctgatgaaccagaccctaaacgccctaaagtggagcccgggggacttggagaccctgttgagggaaacgggctggaccctcctccagatgttgatggaggcggtgtccacctacccggtgatcactaccttgggcctgggaatcctttacaaaatggccccccctctgacatggtggatgcgatcgcccgcgtccacgattaccggtacgcggctatcatggccatgggactcaacccgtacactcactataatatagccgatgccgaattgctcgaaaatcttaaaagccagactggggttcgggccgcggccgcccgagcctatttcgatctgaagaaaatgttattccctcatcttactttacaggccacactcccgggtgttccgagttgggcagcttcacaaacggaatcaaatatgcccggcgacgattctaacaccggaggtgtgggcggcggccaagctaacctcggcgtctgggcccagggggcgaccttcaccgatactgcggtgacctgttacatgacgaggcggtgttacctgccgtttaaccccgacccggtatatgaacccatcacacaccggggccgcaatgcgtataaagagggcccctaccagatggtcatgcgtcaccccgtatacggctacaagaccccctggcattaccctgatttaaacgccttaaacctatacttttccccagccgaatttcaatacttactagaacaatatgagtccattgtgccggcgagcatggacgtactcatcacagatatcattattaaggaagttagccaaatgaacaatacaacccaggtggctgacagcggcactggcgccatagccattctcaccgacgccgattataaatacccctacgtcctagggacggggcaagccaccttgcctccggacattcccacagacatgtatctactgccccaatatgcctatctcaccacagggctcattgagggcgaacaccacgaacaccatgtggccacggacaatcaagtggtcattcctaacagcgacacagatttctatgtgttagaacacagtcactttaccctacaccgcacaggtgacctattttcccacagttatcagtttccggaggccaaacccaaatcactggtgggggccagccaacatttcttcctgatggaaaaccccctctatgactcgaggttaaaagttctagaccgcactggcactaatgccaactggcgacacatcgccaaggaccactatgggtccaaaccccagaacttcttcccaggccccctcatcagttcacataccgcttatgcccatgatggtgttgccccacgatggggcgcgtgggcgtctgagatgcagtggctacagacgggtaccagtgtgggagatgaaccttatgctaggtggtcttttaggcccggccccgcctgccaacctttttcccacacaccagctgacaaccccacagaagaccaaccatctgtgggcgtagacgcggtctcttggggtctacacgaaaacaccagtgtggacacccccggcctgaaccgcgggcggctgcccgatgatcgggaacaggccaaacaactagggggattagatgaaaagttctggccggttacccgcctaaaccaggctcttaaagaagaccaggtttcccacgcacggcccctaatgcccggttcagtgtggaacgaacgggctctccactacgagagtcagatctgggccaaaattccccgctgtgataggagtttcatggctgacagccccatgatgggcggctggggtttcgccgcaccgccgccaatgattttcatcaaaatgttgcccaccccggcgcccctcggtacggacaccgtgaccgaaaatacaacagaaaaggctgggatacttcatcagtacgcagtgtttaaccttaccgtcaagctggtgtttaacttaaagaagcgcaccatgacgggccgctggaacccccagcccgccgtgtaccctccctctgctcggggacacatgccctatgttctgtacaccccccgcgacctcccgggtaatcaacaccctgacgtgtacgggcacgatggctatgagcggtccgaacactgttggaccgctaaagcgcgtgtcgcccacttgtaa

SEQ ID NO: 5 - DNA, 1725 bp, Erythroparvovirus
atgcccggcgacgattctaacaccggaggtgtgggcggcggccaagctaacctcggcgtctgggcccagggggcgaccttcaccgatactgcggtgacctgttacatgacgaggcggtgttacctgccgtttaaccccgacccggtatatgaacccatcacacaccggggccgcaatgcgtataaagagggcccctaccagatggtcatgcgtcaccccgtatacggctacaagaccccctggcattaccctgatttaaacgccttaaacctatacttttccccagccgaatttcaatacttactagaacaatatgagtccattgtgccggcgagcatggacgtactcatcacagatatcattattaaggaagttagccaaatgaacaatacaacccaggtggctgacagcggcactggcgccatagccattctcaccgacgccgattataaatacccctacgtcctagggacggggcaagccaccttgcctccggacattcccacagacatgtatctactgccccaatatgcctatctcaccacagggctcattgagggcgaacaccacgaacaccatgtggccacggacaatcaagtggtcattcctaacagcgacacagatttctatgtgttagaacacagtcactttaccctacaccgcacaggtgacctattttcccacagttatcagtttccggaggccaaacccaaatcactggtgggggccagccaacatttcttcctgatggaaaaccccctctatgactcgaggttaaaagttctagaccgcactggcactaatgccaactggcgacacatcgccaaggaccactatgggtccaaaccccagaacttcttcccaggccccctcatcagttcacataccgcttatgcccatgatggtgttgccccacgatggggcgcgtgggcgtctgagatgcagtggctacagacgggtaccagtgtgggagatgaaccttatgctaggtggtcttttaggcccggccccgcctgccaacctttttcccacacaccagctgacaaccccacagaagaccaaccatctgtgggcgtagacgcggtctcttggggtctacacgaaaacaccagtgtggacacccccggcctgaaccgcgggcggctgcccgatgatcgggaacaggccaaacaactagggggattagatgaaaagttctggccggttacccgcctaaaccaggctcttaaagaagaccaggtttcccacgcacggcccctaatgcccggttcagtgtggaacgaacgggctctccactacgagagtcagatctgggccaaaattccccgctgtgataggagtttcatggctgacagccccatgatgggcggctggggtttcgccgcaccgccgccaatgattttcatcaaaatgttgcccaccccggcgcccctcggtacggacaccgtgaccgaaaatacaacagaaaaggctgggatacttcatcagtacgcagtgtttaaccttaccgtcaagctggtgtttaacttaaagaagcgcaccatgacgggccgctggaacccccagcccgccgtgtaccctccctctgctcggggacacatgccctatgttctgtacaccccccgcgacctcccgggtaatcaacaccctgacgtgtacgggcacgatggctatgagcggtccgaacactgttggaccgctaaagcgcgtgtcgcccacttgtaa

SEQ ID NO: 6 - DNA, 1891 bp, Erythroparvovirus
  CDS (2)..(1891)
gaagttgaccgcgaagctccacagcatgtttgcggaatgcggggcaggcgaggagaccaagtactattacagcgtgccgcgtgaccggcacgggaaagtacttaggtgtgacgaggagttcatccgcacttatttgttgaaaaaggtcccgctagacggtgtgtggtgggcttggacgactattgacaaatttaaacccatagttctcagcgagacccggcgccgctccctggcagccgctgagcggctgaacgacggtgggacagaccaggcgctagtcccggtccaacctccgccggccaagcggacgcgggcttctgacgaatttgtgactttatgcaactggcttgcccaggaaggcattctgaccgaacaaaagtggagagcgactgatcttaacgggtatgtacgacagtatgcctctatgcacggccgtcagcaggtagcggccgcgctgatgatggcccgtaacataatccttgataccatggccaacagccattttctcaccagaggccccccagccgtggtagccaccccggcgtgggaggaaaacagggtggtccaattgttgcgccttcagggctatgatccgcgggtggtggggcagtatattctagcttgggcggacggaatgacggggaagaggaatacactttggctctatgggcctcccagcacgggaaaaacaaatctcgccaatgcgatctgtcagtgcgtgcccgtgtacgggatggttaattggaccaatgaaaattttccatttaatgatgcggcagataaatgtctgattttgtgggatgagggacgcatcagtagtaaaattgtggaggcctgtaaggccatcttgggcggcatgccggttcgagtagaccaaaaatgtaaggggagcaccgccctccggccgaccccggtgttggttaccagcaacggggatatgaccatcgtcagagatggcaataccagtactttgatccatcggcctgccctgaaagatcgcatggtgaggctagacttcaatgtaaagttgcccgctgaatatgggttgttgacccccagtgatgtgcgggcctggttggcaggttgtcgggaccaccccccactggtcgatgcgatcaacatggggtttacttttcctgtgggcagtgatgctcacaaggaggacgagggcgatgacgacgccgaccatagcggcagtcgtctccacagcagtcccttccagtcacgtcgcgtcccggacacccccagtgacggaatctggcccgaggacgacgagtcagactctgacgcggagctctccgagggcgacctcaacgacatcctccccctcgtcaactcctgggaaagttccgtgtccccgacataccggctcgaacgacccatttgtggtgtcgattacgtgtggtcgacccttctcaacggggctgtgtgttcggcggaacacgccacggcggcgaagccgcctccttgcttcgactgctatcgggtgcactccatccttgggtgttgggcggttcgggaccgcgaaaacgggcccgaaaccggcgacctaaggcgttgtgttctacaccgtaacagtaaagatcccctggatgttcttccctgtcaccgctgtacggcactgagtggcttagaacactttgtgtctagcgactctcccccaaaagtgtacggggccgccacggaggtggtggcctttggcgccggtggtcaggaaaccccgaaacaaacgccaactacccagccccctgtggtgactccccctgccccacgccgtccacccgtggttaaagtgcttcccttgtccccccccgctcctccgcctccttcgccggctggcccgagcccttcgccccccccactcaacgtccgtcgtcgtttgagtttctga

SEQ ID NO: 7 - protein, 629 aa, Erythroparvovirus
KLTAKLHSMFAECGAGEETKYYYSVPRDRHGKVLRCDEEFIRTYLLKKVPLDGVWWAWTTIDKFKPIVLSETRRRSLAAAERLNDGGTDQALVPVQPPPAKRTRASDEFVTLCNWLAQEGILTEQKWRATDLNGYVRQYASMHGRQQVAAALMMARNIILDTMANSHFLTRGPPAVVATPAWEENRVVQLLRLQGYDPRVVGQYILAWADGMTGKRNTLWLYGPPSTGKTNLANAICQCVPVYGMVNWTNENFPFNDAADKCLILWDEGRISSKIVEACKAILGGMPVRVDQKCKGSTALRPTPVLVTSNGDMTIVRDGNTSTLIHRPALKDRMVRLDFNVKLPAEYGLLTPSDVRAWLAGCRDHPPLVDAINMGFTFPVGSDAHKEDEGDDDADHSGSRLHSSPFQSRRVPDTPSDGIWPEDDESDSDAELSEGDLNDILPLVNSWESSVSPTYRLERPICGVDYVWSTLLNGAVCSAEHATAAKPPPCFDCYRVHSILGCWAVRDRENGPETGDLRRCVLHRNSKDPLDVLPCHRCTALSGLEHFVSSDSPPKVYGAATEVVAFGAGGQETPKQTPTTQPPVVTPPAPRRPPVVKVLPLSPPAPPPPSPAGPSPSPPPLNVRRRLSF

SEQ ID NO: 8 - DNA, 420 bp, Erythroparvovirus
tcaacccgtacactcactataatatagccgatgccgaattgctcgaaaatcttaaaagccagactggggttcgggccgcggccgcccgagcctatttcgatctgaagaaaatgttattccctcatcttactttacaggccacactcccgggtgttccgagttgggcagcttcacaaacggaatcaaatatgcccggcgacgattctaacaccggaggtgtgggcggcggccaagctaacctcggcgtctgggcccagggggcgaccttcaccgatactgcggtgacctgttacatgacgaggcggtgttacctgccgtttaaccccgacccggtatatgaacccatcacacaccggggccgcaatgcgtataaagagggcccctaccagatggtcatgcgtcaccccgtatacggctaca

SEQ ID NO: 9 - DNA, 21 bp, Artificial
  primer
gacccggtatatgaacccatc

SEQ ID NO: 10 - DNA, 20 bp, Artificial
  Primer
gacgcatgaccatctggtag

SEQ ID NO: 11 - DNA, 20 bp, Artificial
  Primer
gagcctatttcgatctgaag

SEQ ID NO: 12 - DNA, 22 bp, Artificial
  Primer
aatgttattccctcatcttact

SEQ ID NO: 13 - DNA, 20 bp, Artificial
  Primer
catgtaacaggtcaccgcag

SEQ ID NO: 14 - DNA, 20 bp, Artificial
  Primer
cagacgccgaggttagcttg

SEQ ID NO: 15 - protein, 575 aa, Artificial
  pcDNA3.1 plasmid
MAPGDDSNTGGVGGGQANLGVWAQGATFTDTAVTCYMTRRCYLPFNPDPVYEPITHRGRNAYKEGPYQMVMRHPVYGYKTPWHYPDLNALNLYFSPAEFQYLLEQYESIVPASMDVLITDIIIKEVSQMNNTTQVADSGTGAIAILTDADYKYPYVLGTGQATLPPDIPTDMYLLPQYAYLTTGLIEGEHHEHHVATDNQVVIPNSDTDFYVLEHSHFTLHRTGDLFSHSYQFPEAKPKSLVGASQHFFLMENPLYDSRLKVLDRTGTNANWRHIAKDHYGSKPQNFFPGPLISSHTAYAHDGVAPRWGAWASEMQWLQTGTSVGDEPYARWSFRPGPACQPFSHTPADNPTEDQPSVGVDAVSWGLHENTSVDTPGLNRGRLPDDREQAKQLGGLDEKFWPVTRLNQALKEDQVSHARPLMPGSVWNERALHYESQIWAKIPRCDRSFMADSPMMGGWGFAAPPPMIFIKMLPTPAPLGTDTVTENTTEKAGILHQYAVFNLTVKLVFNLKKRTMTGRWNPQPAVYPPSARGHMPYVLYTPRDLPGNQHPDVYGHDGYERSEHCWTAKARVAHL

SEQ ID NO: 16 - protein, 589 aa, Artificial
  pcDNA3.1 plasmid Myc-tag
MAPGDDSNTGGVGGGQANLGVWAQGATFTDTAVTCYMTRRCYLPFNPDPVYEPITHRGRNAYKEGPYQMVMRHPVYGYKTPWHYPDLNALNLYFSPAEFQYLLEQYESIVPASMDVLITDIIIKEVSQMNNTTQVADSGTGAIAILTDADYKYPYVLGTGQATLPPDIPTDMYLLPQYAYLTTGLIEGEHHEHHVATDNQVVIPNSDTDFYVLEHSHFTLHRTGDLFSHSYQFPEAKPKSLVGASQHFFLMENPLYDSRLKVLDRTGTNANWRHIAKDHYGSKPQNFFPGPLISSHTAYAHDGVAPRWGAWASEMQWLQTGTSVGDEPYARWSFRPGPACQPFSHTPADNPTEDQPSVGVDAVSWGLHENTSVDTPGLNRGRLPDDREQAKQLGGLDEKFWPVTRLNQALKEDQVSHARPLMPGSVWNERALHYESQIWAKIPRCDRSFMADSPMMGGWGFAAPPPMIFIKMLPTPAPLGTDTVTENTTEKAGILHQYAVFNLTVKLVFNLKKRTMTGRWNPQPAVYPPSARGHMPYVLYTPRDLPGNQHPDVYGHDGYERSEHCWTAKARVAHLSALEEQKLISEEDL

(19) United States
(12) Patent Application Publication
(10) Pub. No.: US 2023/0340032 A1
(43) Pub. Date: Oct. 26, 2023

(54) NOVEL ERYTHROPARVOVIRUS ASSOCIATED WITH RESPIRATORY DISTRESS IN EQUINE

(71) Applicant: Intervet Inc., Madison, NJ (US)

(72) Inventors: Ad De Groof, Holten (NL); Cornelia Maria Van Der Hoek, Amsterdam (NL); Martin Deijs, Huizen (NL); Cornelis Maanen Van, Holten (NL)

(73) Assignee: Intervet Inc., Madison, NJ (US)

(21) Appl. No.: 17/800,608

(22) PCT Filed: Feb. 22, 2021
(86) PCT No.: PCT/EP2021/054329

§ 371 Date: Aug. 18, 2022

(30) Foreign Application Priority Data
Feb. 21, 2020 (EP) 20158648.4

(51) Int. Cl. C07K 14/015; C12N 7/00; A61K 39/23; C12Q 1/70

(52) U.S. Cl. CPC C07K 14/015; C12N 7/00; A61K 39/23; C12Q 1/701; A61K 2039/5254

(57) ABSTRACT

The present invention relates to a novel equine parvovirus associated with respiratory distress and recurrent laryngeal neuropathy in equine, to nucleic acid fragments and proteins of the virus and to vaccines based upon the virus, proteins and nucleic acid fragments. The invention also relates to a diagnostic test for the detection of the vims or antibodies against the virus.

### FIELD OF THE INVENTION

The invention in general pertains to a new virus which is a member of the sub-family Parvovirinae of the family of the Parvoviridae, the virus being associated with a disorder in equine. The invention also pertains to subunits of this novel virus, in particular nucleic acid fragments corresponding to the new virus and proteins of this virus, as well as to vaccines for combatting the virus and a diagnostic test kit for diagnosing the presence of the virus in a host animal.

### BACKGROUND OF THE INVENTION

Over the last years, an increase is seen in the number and the size of farms where foals of similar age are raised in group housing to three-year-olds, the age at which horses usually return to their owners to be trained for sports and leisure activities, at which horses are clinically examined, and at which studbook inspection is conducted. As is known from animal husbandry in general, large numbers of animals living closely together are at increased risk for attracting all kinds of diseases, even diseases hardly known or seen or even unknown before the days of large-scale commercial farming.

In the last couple of years, outbreaks of a clinical disease syndrome in horses in group housing have been described in several countries, including The Netherlands, Sweden, Belgium, and the United Kingdom. The clinical disease syndrome comprises a sudden onset of severe respiratory distress, sometimes associated with bleedings from the nostrils, nasal cavity, or nasopharynx (epistaxis, left and right), particularly after excitation. Some horses die from asphyxiation. Thus, the clinical symptoms show a sudden onset that may be triggered by excitation and aggravated by exercise.

Upon laryngeoscopic examination, non-purulent mucus and swollen mucosae (edema) are often noticed. Different degrees of total or hemi-laryngeal paralysis are present in all clinical cases. The (bilateral) laryngeal paralysis results in an inability of the laryngeal muscles to regulate arytenoid movement, which is vital for enabling unimpeded inspiratory airflow. Furthermore, this paralysis results in an inability to move the vocal cords. The acute symptoms are in some cases reported to be preceded or followed by a mild form of ataxia. Especially the cases with bilateral paralysis appear to be fatal. Fatality estimates are about 25% (range estimate 10-32% based on 3 outbreaks). It appears that at least some surviving horses are slowly improving slightly over time, but the paralysis persists at least during the first 2 months after onset of symptoms, with severe impact on the horse ability to exercise. The syndrome was observed in 2- and some 3-year-old horses in The Netherlands, but in other countries also in breeding mares or middle-aged sport horses.

In groups where these dramatic cases, including epistaxis and dyspnea, were diagnosed, it was observed that other horses in the group with limited or no clinical symptoms also showed unilateral or bilateral laryngeal paralysis as described above. The etiology of the observed acute respiratory distress, leading to epistaxis and dyspnea in severe case, related to (bilateral) laryngeal paralysis, and possible ataxia henceforward referred to as ‘the disease’ is unclear. In contrast with unilateral laryngeal paralysis, bilateral laryngeal paralysis is rare in equine species.

Laryngeal paralysis and partial paralysis (paresis) has already been recognised as an incurable disease of horses by Fleming and Cadiot in the nineteenth century. The most common type of equine laryngeal paralysis is described in literature as recurrent laryngeal neuropathy (RLN), which is also referred to as idiopathic laryngeal hemiplegia (ILH) because of the unknown etiology. This disorder is an axonopathy characterised by a distal degeneration of the recurrent laryngeal nerve (left more than right) with no known cause. RLN is a mononeuropathy, as other peripheral nerves of the horse remain unaffected, and is unilateral in the majority of cases. Clinically, RLN presents a roaring noise during exercise, with sometimes gasping for breath after exercise and exercise intolerance.

RLN has been described to occur in horses from a few months old to 10 years of age and older, with large-breed horses (such as thoroughbreds and draught breeds) more commonly affected than small-breed horses or ponies. The incidence is highest in young horses, often diagnosed before they have started any type of training, or in 2- and 3-year-olds that are racing or are in race training. The prevalence of RLN varies between breeds, with the largest population studied being the Thoroughbred, where between 2.6% and 8% of horses are reported to be affected. However, in the heavy draught breeds, an incidence of up to 35% has been reported. RLN thus most commonly affects larger horses, usually the left side of the larynx and, much less frequently, the right side or bilaterally. The average age of onset typically ranges from 2-12 years. Studies on RLN are complicated by the fact that RLN-associated paralysis is also observed in clinically unaffected horses. This presence of many subclinical cases means that case selection for “unaffected” controls for research is challenging.

Besides by RLN, damage to the recurrent laryngeal nerves can be induced by other conditions, such as guttural pouch mycosis. Furthermore, the recurrent laryngeal nerves can be damaged as a result of perivascular jugular vein injection, trauma from injuries or surgical procedures of the neck, strangles abscessation of the head and neck, and impingement by neoplasms of the neck or chest. These conditions can result in unilateral and, with usually sudden onset, complete laryngeal paralysis (i.e. hemiplegia).

Importantly, the unusual condition of bilateral laryngeal paralysis has been recorded in horses following organophosphate poisoning, hepatic encephalopathy and following general anesthesia. Plant poisoning, lead toxicity, and central nervous system diseases have also been shown to cause laryngeal paralysis.

The one-sided nature of the laryngeal paralysis, age of onset and relatively mild character of RLN clinical symptoms as reported in literature at first glance appear to be different when compared to the disease related to the present invention. However, the disease in relation to this invention shows characteristics with regard to laryngeal paralysis similar to those seen in RLN, organophosphate poisoning, plant poisoning, lead toxicity, central nervous system disease, hepatic encephalophaty and following general anesthesia, although there is a tendency of a higher percentage of bilateral paralysis. Still the fact that non-affected horses in an affected group, i.e. horses having limited or no clinical symptoms, also showed unilateral or bilateral laryngeal paralysis, it might thus be that the current disease is a more severe and peracute presentation of a disease that is already recognised in the field, viz. RLN, although it may also be an entirely new disease.

Very recently the disorder which is the subject of the present invention was reported about in the Dutch newspaper “De Paardenkrant”. In the paper of 12 Feb. 2020, the front page has an article regarding recent outbreaks of respiratory distress in young horses, referring to a mysterious disease in foal rearing which causes laryngeal paralysis and respiratory distress. It was described that the cause of the disorder was not found.

Therefore, it remains unclear what factors play a role in the origin of the disease. It may be that the disease is caused by a single infectious agent, or that an infectious agent is required as a factor in combination with a pre-existing condition. For example, it may be that the disease, like many other animal diseases, is a multifactorial disease in which besides stress, genetic predisposition and several environmental and management factors, an infectious agent plays a role.

### OBJECT OF THE INVENTION

It is an object of the invention to provide a factor associated with respiratory distress in equine, thus enabling adequate diagnosing and potentially treatment of the corresponding disease.

### SUMMARY OF THE INVENTION

It was found that a virus which is a member of the sub-family Parvovirinae of the family of the Parvoviridae is associated with the disease. The new virus is an erythro-parvovirus, and has a viral genome (partial genome encompassing about 4.8 Kb) comprising a nucleotide sequence which has a level of identity of at least 70% to the nucleotide sequence as depicted in SEQ ID NO: 1. Thus, the level of identity may for example be 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%. A suitable program for the determination of a level of identity is the nucleotide blast program (blastn) of NCBI's Basic Local Alignment Search Tool, using the “Align two or more sequences” option and standard settings (http://blast.ncbi.nlm.nih.gov/Blast.cgi). The virus can be isolated from the blood of affected horses and from nasal swabs. For the purpose of this invention isolated means set free from tissue with which the virus is associated in nature. An example of an isolated virus is the virus as present in cell culture.

It is one of the merits of the present invention that it is now for the first time possible to follow the course of the viral infection associated with RD, and to analyse the presence or absence of the novel virus in the various organs and body fluids of horses suffering from RD. This helps to gain more insight in the development of disease. It is known that in the weeks or even days before an individual horse shows full clinical signs of RD, no abnormalities are observed. The timing between the first symptoms and death of the animals is variable, this can be very short, within a day but it can also take up to a month or longer. It is another merit of the present invention that it is now possible to infect healthy horses with the novel virus and to examine the route of viral infection. It is also one of the merits of the present invention that since the novel equine parvovirus has now been isolated and associated with RD, the virus and/or protective subunits of the virus can be used as the starting material for vaccination purposes.

The novel virus was found by taking and analysing serum and nasal swab samples from affected horses and search for the presence of viruses. Surprisingly a hitherto unknown virus was found in 80-90% of the clinically affected horses, which shows that this new virus is at least associated with the disease. The fact that the virus was not detected in all animals may be explained by the interval between onset of the clinical symptoms and the moment the serum samples and/or swabs were taken (varying between days, weeks, even months). Viremia typically does not exist during all stages after infection and onset of the disease. This is confirmed for the current disease i.a. by the fact that the amounts of virus found per animal varied to a great extent. It is contemplated by the inventors that in the 10-20% of the horses in which the virus is seemingly absent, this is likely due to the fact that the amount of virus present in those horses was below the detection level at the moment of analysis. Furthermore, the site of initial virus replication is not known for this novel virus, and thus the primary site of virus replication after infection may not have been sampled. Still, showing presence of the virus in 80-90% of the affected horses (without knowing the site of initial virus replication and the timing for viremia) is so high that it is beyond reasonable doubt that the novel virus is associated with the disease.

Since the novel virus was detected in horses suffering from acute respiratory distress, leading to symptoms such as epistaxis and dyspnea, and (bilateral) paralysis, the virus will be further referred to as RD-associated virus (abbreviated RDAV). (Acute) respiratory distress can now be characterised by the presence of the novel virus according to the invention at some stage during the disease in organs of animals suffering from RD-induced dyspnea, in severe cases in combination with the following clinical symptoms: acute respiratory distress, epistaxis, edema in the larynx with nonpurulent mucus, swollen mucosae and clotted blood in the nose. Based on a limited number of post-mortem examinations, edema in the lung and bleedings in the lung can be regarded as symptoms of the RD-induced dyspnea.

The nucleotide sequence of the novel viral genome was analysed and revealed that the novel virus belongs to the genus of Erythroparvoviruses of the Parvovirinae subfamily within the Parvoviridae. Parvoviruses are linear, non-segmented single-stranded DNA viruses, with an average genome size of about 5000 nucleotides (5 Kb) and a size in the range of 18-26 nm in diameter.

The novel virus comprises two large Open Reading Frames (ORFs): ORF1 encoding nonstructural protein 1 (NS1), and ORF2 encoding the capsid protein VP1. ORF2 appears to encode shorter capsid proteins that are initiated from alternative start codons.

The partial NS1 protein consisting of 629 amino acids is found at position 2-1891 of SEQ ID NO: 6. SEQ ID NO: 7 represents the partial amino acid sequence of the nonstructural protein NS1. NS1 is a member of a superfamily of viral helicases, a pleiotropic nuclear phosphoprotein and required for viral replication. It is a multi-functional protein that has a role in control of cellular transcription, virus replication, induction of cell death, and transactivation of cellular promoters, and it may have a role in the induction of auto-immune disorders and the induction of cytokines. Therefore, it may be suitable as a target for vaccination.

ORF2 encoding Capsid Protein (CP) VP1 is found at position 1-2679 of SEQ ID NO: 4. VP1 consists of 892 amino acids and is the translation of nucleotides 1-2679 of SEQ ID NO: 4.

VP2 is a shorter protein in the same reading frame as VP1 that uses an alternative start and is the translation of nucleotides 955-2679 of SEQ ID NO: 4 and consists of 574 amino acids. SEQ ID NO: 5 represents the nucleotide sequence of VP2.

VP3 is an even shorter protein in the same reading frame as VP1 that uses an alternative start and is the translation of nucleotides 1060-2679 of SEQ ID NO: 4 and consists of 539 amino acids. SEQ ID NO: 2 represents the nucleotide sequence of VP3. SEQ ID NO: 3 represents the amino acid sequence of the Capsid protein VP3.

The sub-family of the Parvovirinae currently comprises 8 genera (see Cotmore, S. F., Agbandje-McKenna, M., Canuti, M., Chiorini, J. A., Eis-Hubinger, A, Hughes, J., Mietzsch, M., Modha, S., Ogliastro, M., Pénzes, J. J., Pintel, D. J., Qiu, J., Soderlund-Venermo, M., Tattersall, P., Tijssen P., and ICTV Report Consortium, 2019, ICTV Virus Taxonomy Profile: Parvoviridae, Journal of General Virology, 100: 367-368):
1) Amdoparvovirus
2) Aveparvovirus
3) Bocaparvovirus
4) Copiparvovirus
5) Dependoparvovirus
6) Erythroparvovirus
7) Protoparvovirus
8) Tetraparvovirus

At this moment, a number of different parvoviruses have been identified that infect horses.

In 2015 the equine parvovirus CSF was found. The GenBank accession number is KR902500. The virus was found in a sample of a horse showing lymphocytosis and neurological signs of disease. Phylogenetic analysis on the basis of the partial NS1 protein sequences shows that horse parvovirus CSF was most closely related to viruses in the genus Copiparvovirus, with amino acid identity of 29.2— 30.1%. Based on the criteria of The International Committee on Taxonomy of Viruses horse parvovirus EqPV-CSF was classified as a tentative new species in the genus Copiparvovirus, which currently comprises parvoviruses infecting pigs and cows.

In 2018 another equine parvovirus was found, the genome of which is available in GenBank via accession no. MG136722. This virus is also a Copiparvovirus. Suggestions are made in literature that this virus is related to Theiler's disease (more than equine pegivirus, which in literature is often regarded as the causative agent, without experimental proof).

Altan et al. in 2019 (Viruses 2019, 11, 942) found a novel equine Parvovirus. This virus is also a Copiparvovirus, named eqcopivirus. The virus was detected in the plasma of a horse with neurological symptoms. The GenBank accession numbers are MN181466 to MN MN181468. Altan et al. compared 13 samples from horses with neurological signs, horses with respiratory signs and 41 samples from healthy horses. They concluded that based on viral prevalence in plasma samples, none of the three currently genetically characterised equine parvoviruses, all of which were in the copiparvovirus genus, was significantly associated with neurological and respiratory signs in this limited sampling.

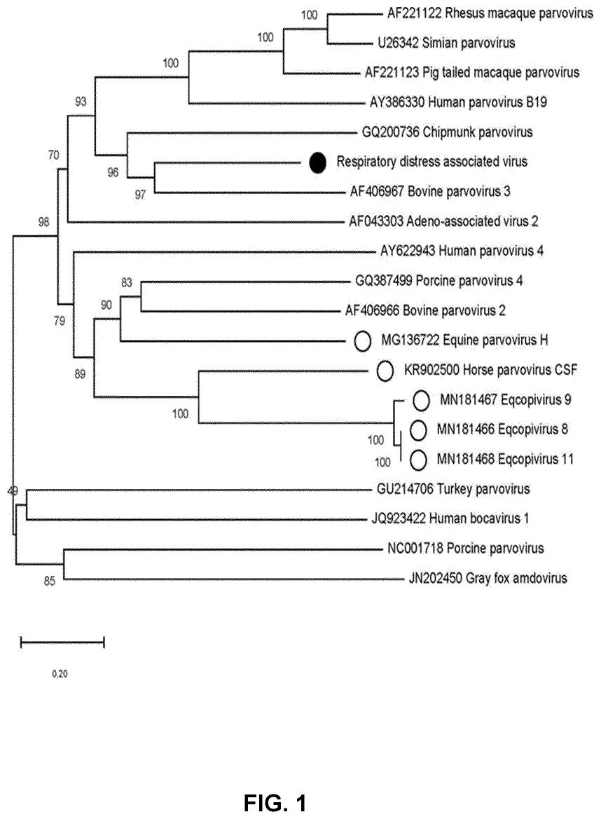

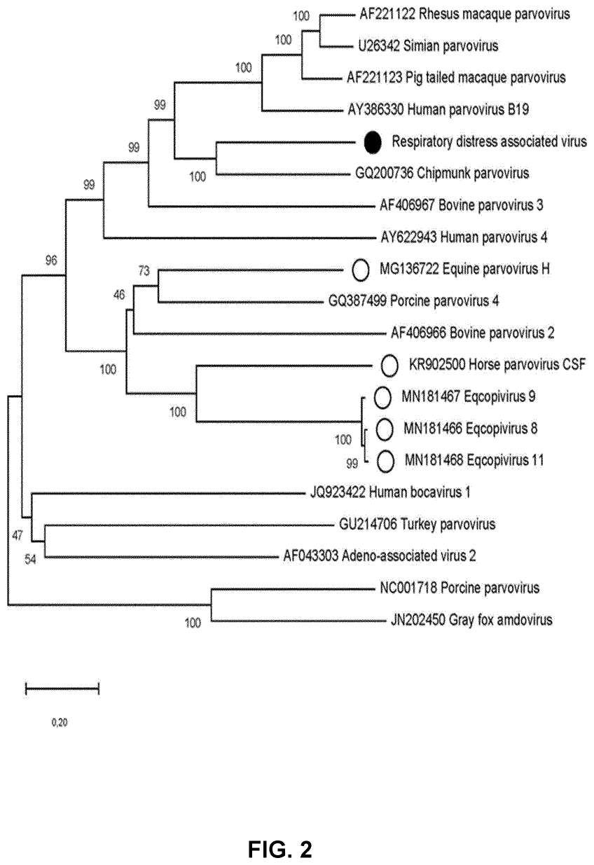

The phylogenetic tree of the present partial NS1 protein is presented in FIG. 1, that of ORF2-encoded VP1 protein in FIG. 2. As follows from these phylogenetic trees, the novel equine parvovirus is related to the sub-family of Erythroparvoviruses, such as Chipmunk parvovirus and the human parvovirus B19. For this reason, the novel virus is placed in the group of the Erythroparvoviruses. SEQ ID NO: 6 (NS1) and SEQ ID NO:4, and 2 (CPs) show low-level identity to the characterised homologous proteins in Chipmunk parvovirus and Human parvovirus B19. In Human parvovirus B19, the viral capsid is formed by 60 subunits composed of ˜95% VP2 of 554 amino acids and -5% VP1, which is cocarboxyterminal with VP2 but has an N-terminal extension of 227 amino acids. It can be deducted from SEQ ID NO: 4, SEQ ID NO: 5 and SEQ ID NO: 2 that a similar putative N-terminal extension of capsid proteins exists in the RD-associated virus.

It will be understood that for the CP and NS1 proteins natural variations can exist between individual representatives of the novel RD-associated virus. Genetic variations leading to minor changes in e.g. the Capsid Protein sequence do exist. This is equally true for the NS1 gene. First of all, there is the so-called “wobble in the second and third base” explaining that nucleotide changes may occur that remain unnoticed in the amino acid sequence they encode: e.g. triplets TTA, TTG, TCA, TCT, TCG and TCC all encode Leucine. In addition, minor variations between representatives of the novel equine parvovirus according to the invention may be seen in amino acid sequence.

These variations can be reflected by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science 227, 1435-1441, 1985) and determining the functional similarity between homologous proteins. Such amino acid substitutions of the exemplary embodiments of this invention, as well as variations having deletions and/or insertions are within the scope of the invention.

This explains why the Capsid Protein and the nonstructural protein NS1, when isolated from different representatives of an equine parvovirus according to the invention, may have sequence identity levels that are significantly below 100%, while still representing the Capsid Protein and the nonstructural protein NS1 of the equine parvovirus according to the invention. This is clearly reflected e.g. in the phylogenetic tree in FIG. 1 of a paper by Xiao et al., Veterinary Microbiology 161: 325-330 (2013) where it is shown that even within one single genus, the PARV4-like virus genus, consisting of highly related parvoviruses nevertheless have significantly different overall genomic nucleotide sequences as well as significantly different NS1 gene nucleotide sequences. As is commonly known in the art of recombinant expression of proteins, sequence optimization of a nucleic acid sequence can lead to a variation of over 50% with the natural nucleic acid, while still coding for the corresponding wild type protein or a functionally equivalent protein.

### EMBODIMENTS OF THE INVENTION

The invention is also embodied in a cell culture comprising the novel Erythroparvovirus. Cell cultures of Erythroparvoviruses are commonly known and have been described elaborately (e.g. Ozawa et al., 1987. Blood 70:384-391; Ozawa et al., 1986. Science 233:883-886). Wong et al. (2008. J. Virol. 82:2470-76) have described ex vivo-generated CD36+ erythroid progenitors that are highly permissive to human parvovirus B19 replication, which is an Erythroparvovirus closely related to the virus of the present invention. Other examples of cells and cell lines are E. Derm (ATCC CCL57, https://www.atcc.org/products/all/CCL-57.aspx, Immortalised Equine Lung Cells (extEqFL), https://www.abmgood.com/Immortalised-Equine-Lung-Cells-(extEqFL)-T0095.html, FHK-78 cells, FHK-Tcl3.1 9 (Oguma et al., J. Vet. Med. Sci. 75(9): 1223-1225, 2013) and ETCC cells (Allen et al., 1974. Am. J. Vet. Res. 35: 1153-1160 . SK6, PK15, primary or immortalised equine kidney cells, primary or immortalised respiratory epithelial cells, and primary or immortalised equine alveolar lung macrophages. Almost the whole viral genome of the novel equine parvovirus has now been determined and the DNA sequence of a representative of the novel virus is presented in SEQ ID NO: 1. Here beneath (Example 7) it is indicated how the whole genome can be obtained. Parvoviruses by definition belong to the smallest viruses known, the whole ss-DNA encoding the parvovirus according to the invention can be made synthetically. Cloning of full-length parvoviral DNA into a plasmid such as e.g. Bluescript II SK, and the subsequent generation of whole parvovirus through transfection of equine cells with an expression plasmid encoding the novel equine parvovirus is i.a. described by Qiu et al., J. Virol. 79: 11035-11044 (2005) and by Wang et al., J. Virol. Meth. 200: 41-46 (2014).

A permissive cell line such as primary or immortalised equine kidney cells, primary or immortalised respiratory epithelial cells, and primary or immortalised equine alveolar lung macrophages would be the cell line of first choice for this purpose. Nevertheless, if desired non-permissive cell lines can also be used, for example by replication with the help of adenovirus genes as described by Guan et al., J. Virol. 83: 9541-9553, 2009 (see Example 7).

The invention is also embodied in a nucleic acid fragment comprising a gene encoding a capsid protein (CP), wherein the said gene has a nucleotide sequence that has a level of identity of at least 70% to the nucleotide sequence of the CP gene as depicted in SEQ ID NO: 2. This gene encodes the CP of the novel Erythrorparvovirus which is associated with RD in equine. The level of identity may be higher than 70%, for example 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%. The length of the nucleic acid fragment preferably is at least 50% of the length of the CP gene of SEQ ID NO: 2, more preferably at least 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%. The nucleic acid fragment can be a DNA or an RNA fragment, as both can have their use in treatment and diagnostics. Although the sequence provides the DNA nucleotides, it is common to define the corresponding RNA with the same nucleotide sequence.

The invention is also embodied in the corresponding protein, i.e. the CP encoded by a nucleic acid fragment as defined here above. Alternatively, the invention is embodied in a CP comprising an amino acid sequence which has a level of identity of at least 70% to the amino acid sequence according to SEQ ID NO: 3. Thus, the level of identity may for example be 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%. The length of the amino acid sequence of the CP of this embodiment preferably is at least 50% of the length of the CP as depicted in SEQ ID NO: 3, more preferably at least 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%.

The invention is also embodied in a nucleic acid fragment comprising a gene encoding a non-structural protein 1 (NS1), wherein the said gene has a nucleotide sequence that has a level of identity of at least 70% to the nucleotide sequence of the (partial) NS1 gene as depicted in SEQ ID NO: 6. This gene encodes the NS1 of the novel Erythrorparvovirus which is associated with RD in equine. The level of identity may be higher than 70%, for example 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%. The length of the nucleic acid fragment preferably is at least 50% of the length of the NS1 gene of SEQ ID NO: 6, more preferably at least 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%. The nucleic acid fragment can be a DNA or an RNA fragment, as both can have their use in treatment and diagnostics. Although the sequence provides the DNA nucleotides, it is common to use the same sequence also for defining the corresponding (complementary) RNA.

The invention is also embodied in the corresponding protein, i.e. the NS1 encoded by a nucleic acid fragment as defined here above. Alternatively, the invention is embodied in a NS1 comprising an amino acid sequence which has a level of identity of at least 70% to the amino acid sequence according to SEQ ID NO: 7. Thus, the level of identity may for example be 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%. The length of the NS1 protein according to this embodiment preferably is at least 50% of the length of the NS1 \protein according to SEQ ID NO: 7, more preferably at least 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or even 100%.

The invention is further embodied in a vaccine for combating (an infection with) RD-associated Erythroparvovirus in equine, wherein said vaccine comprises an immunogenically effective amount of the novel virus and a pharmaceutically acceptable carrier. The virus may be in a live attenuated form or in an inactivated form. Examples of pharmaceutically acceptable carriers are commonly known in the art and may for example be sterile water, saline, aqueous buffers such as PBS and the like. In addition, a vaccine according to the invention may comprise other additives such as adjuvants (e.g. vitamin E, non-ionic block polymers, muramyl dipeptides, Quill A, mineral oil such as Bayol® or Marcol®, vegetable oil, squalene, squalene, Carbopol®, aluminum salts such as aluminum hydroxide, etc.), stabilizers, anti-oxidants and others. “Combating” in this respect should be interpreted in a broad sense. Combating RD is considered to comprise vaccination in order to prevent, ameliorate or cure an infection with the Erythroparvovirus, or any sign of the disease (RD) or another disorder that is associated with this infection. Vaccination may take place before the initial infection (prophylactic vaccination) or once the virus is diagnosed in an infected animal that is not yet suffering from the syndrome (therapeutic vaccination). In practice, vaccination in a herd of animals will often be a mix of prophylactic and therapeutic vaccination.

Attenuated live virus vaccines, i.e. vaccines comprising the virus according to the invention in a live attenuated form, have the advantage over inactivated vaccines that they best mimic the natural way of infection. In addition, their replicating abilities allow vaccination with low amounts of viruses; their number will automatically increase until it reaches the trigger level of the immune system. From that moment on, the immune system will be triggered and will finally eliminate the viruses. A live attenuated virus is a virus that has a decreased level of virulence when compared to virus isolated from the field. A virus having a decreased level of virulence is considered a virus that even in combination with other factors involved in RD does not induce mortality in horses.

As is commonly known, attenuated parvoviruses can e.g. be obtained by growing the viruses according to the invention in the presence of a mutagenic agent, followed by selection of virus that shows a decrease in progeny level and/or in replication speed. Many such agents are known in the art. Another very often used method is serial in vitro passage. Viruses then get adapted to the cell line used for the serial passage, so that they behave attenuated when transferred to the natural host again as a vaccine. Still another way of obtaining attenuated viruses is to subject them to growth under temperatures deviating from the temperature of their natural habitat. Selection methods for temperature sensitive mutants (Ts-mutants) are well-known in the art. Such methods comprise growing viruses in the presence of a mutagen followed by growth at a sub-optimal temperature and at the optimal temperature, titration of progeny virus on cell layers and visual selection of those plaques that grow slower at the optimal temperature. Such small plaques comprise slow-growing and thus desired live attenuated viruses.

Live attenuated vaccines for combatting parvovirus infection have been described i.a. by Paul & Mengeling, Am. J. Vet. Res.: 41: 2007-2011 (1980), by Paul & Mengeling, Am. J. Vet. Res.: 45: 2481-2485 (1984) and by Fujisaki e & Murakami, Nat. Inst. of Animal Health quarterly (Tokyo) 22: 36-37 (1982). Such live attenuated virus vaccines can equally be made for the novel equine parvovirus according to the invention.

A possible disadvantage of the use of live attenuated viruses however might be that inherently there is a certain level of virulence left. This is not a real disadvantage as long as the level of virulence is acceptable, i.e. as long as the vaccine at least prevents the horses from dying. Of course, the lower the rest virulence of the live attenuated vaccine is, the less influence the vaccination has on weight gain during/after vaccination.

Inactivated vaccines are, in contrast to their live attenuated counterparts, inherently safe, because there is no rest virulence left. In spite of the fact that they usually comprise a somewhat higher dose of viruses compared to live attenuated vaccines, they may e.g. be the preferred form of vaccine in horses that are suffering already from other diseases. Horses that are kept under sub-optimal conditions, such as incomplete nutrition or sub-optimal housing would also benefit from inactivated vaccines.

Therefore, another embodiment of the present invention relates to a vaccine comprising a virus according to the invention wherein said virus is in an inactivated form.

It is known that whole inactivated parvoviruses in general, be it porcine or canine parvoviruses, are a very efficient and safe basis for vaccines. Merely as an example: MSD AH (Boxmeer, The Netherlands) produces a commercially available inactivated parvovirus type PPV vaccine, viz. Porcilis® Parvo. Hipra (Spain) also produces a commercially available inactivated equine parvovirus type PPV vaccine, viz. PARVOSUIN® MR/AD. Zoetis produces an inactivated Canine parvovirus, viz. PARVAC®, and an inactivated equine parvovirus type PPV vaccine, viz. Equine PARVAC®. Novartis provides methods for the inactivation of parvovirus in U.S. Pat. No. 4,193,991.

Such inactivated whole virus vaccines can equally be made for the novel equine parvovirus according to the invention. As is the case for known parvovirus vaccines, the production basically comprises the steps of growing the novel parvovirus on susceptible equine cells, harvesting the virus, inactivating the virus and mixing the inactivated virus with a pharmaceutically acceptable carrier.

The standard way of inactivation is a classical treatment with formaldehyde. Other methods well-known in the art for inactivation are UV-radiation, gamma-radiation, treatment with binary ethylene-imine, thimerosal and the like. The skilled person knows how to apply these methods. For example, the virus is inactivated with β-propiolactone, glutaraldehyde, ethylene-imine or formaldehyde. However, other ways of inactivating the virus are also embodied in the present invention.

The invention is also embodied in a vaccine for combating (an infection with) RD-associated Erythroparvovirus in equine, wherein said vaccine is a subunit vaccine comprising an immunogenically effective amount of a Capsid Protein as defined hereabove and/or an immunogenically effective amount of a non-structural protein 1 as defined here above, and a pharmaceutically acceptable carrier. Although whole inactivated parvoviruses provide a good basis for vaccines, their production may be expensive, depending i.a. upon the type of host cells used, the substrate and the cell culture medium used.

In the specific case of parvoviruses, an attractive alternative for the use of whole viruses is the use of parvovirus CP or NS1 subunits. Such subunits, in particular the CP subunits may be present in the vaccine in the form of so-called empty capsids.

Such empty capsids are basically virus-like particles that however do not comprise the parvoviral genome. As a consequence, parvoviral empty capsid particles do not have to be inactivated before use in a vaccine, and therefore they have the additional advantage that they are intrinsically safe. Empty capsids can be obtained by mere expression of ORF2 encoding the Capsid Protein (VP1 and/or VP2 and/or VP3), in a suitable expression system. The so-formed capsid protein typically self-assembles into empty virus particles. As is commonly known, parvoviral empty capsids can readily be made in large amounts and they are highly immunogenic.

By far most expression systems currently in use for making parvoviral empty capsids are baculovirus-based expression systems. Methods for the production of highly immunogenic parvovirus empty capsids in baculovirus-based expression systems have been e.g. described for porcine parvovirus type PPV by Martinez et al., Vaccine 10: 684-690 (1992), Casal et al., Biotechnology and Applied Biochemistry 29: 141-150 (1999), Zhou et al., Virology Journal 7: 366 (2010) and by Hao Feng et al., PlosOne; Jan. 17, 2014, DOI: 10.1371/journal.pone.0079575. For other parvoviruses, such methods have been described e.g. by Saliki et al., Journ. Gen. Virol. 73: 369-74 (1992) and by Brown et al., Journal of Virol. 65: 2702 (1991). Furthermore, baculovirus expression systems and baculovirus expression vectors in general have been described extensively in textbooks such as by O'Reilly et al., Baculovirus Expression Vectors, A Laboratory Manual. By David R. O'Reilly, Lois K. Miller, and Verne A. Luckow. Publisher: Oxford University Press, USA ISBN-10: 0195091310 (Sep. 23, 1993), ISBN-13: 978-0195091311 (May 1994) and Murhammer, 2007: Baculovirus and Insect Cell Expression Protocols. In: Methods in Molecular Biology™, Volume 388 (2007). Editors: David W. Murhammer. ISBN: 978-1-58829-537-8 (Print) 978-1-59745-457-5 (Online).

Baculovirus-based expression systems are also commercially available, e.g. from Invitrogen Corporation, 1600 Faraday Avenue, Carlsbad, California 92008, USA.

An alternative for Baculovirus-based expression systems are yeast-based expression systems. Yeast expression systems are e.g. described by Gellissen (2005), Production of recombinant proteins: novel microbial and eukaryotic expression systems. Editor: Gerd Gellissen, ISBN: 3-527-31036-3. Ready-to-use expression systems are i.a. commercially available from Research Corp. Technologies, 5210 East Williams Circle, Suite 240, Tucson, AZ 85711-4410 USA. Yeast and insect cell expression systems are also e.g. commercially available from Clontech Laboratories, Inc. 4030 Fabian Way, Palo Alto, California 94303-4607, USA.

Expression of the CP or NS1 is of course also possible in mammalian cell based expression systems as known in the art. The amount of subunits such as empty capsids in a vaccine and the route of administration would be comparable with that of inactivated whole virus particles, since in terms of immunogenicity they are comparable to inactivated whole virus particles. Usually, an amount of between 1 and 100 μg of the novel parvovirus subunits (e.g. an empty capsid) would be suitable as a vaccine dose. From a point of view of costs, a preferred amount would be in the range of 1-50 μg, more preferred in the range of 1-25 μg. It is known, i.a. from Casal et al., Biotechnology and Applied Biochemistry 29: 141-150 (1999) that for both canine parvovirus and porcine parvovirus doses as low as 1-3 μg in the presence of conventional adjuvants confer total protection on the corresponding host against the corresponding infection and disease. It is thus understood that also for the present virus, a dose in this range (above 1 μg) is immunogenically effective. Casal successfully used i.a. aluminum hydroxide and Quill A in his parvovirus vaccines.

An alternative to the inactivated whole virus vaccine approach and the subunit vaccine approach is the use of live recombinant non-parvovirus vectors that have horses as their host animal, as carriers of the novel equine parvoviral Capsid Protein or non-structural protein 1 gene. Amongst a suitable recombinant non-parvovirus vectors that has horses as its host animal is Equine herpesvirus. Adeno virus or pox virus vectors may be equally suitable.

The invention is also embodied in a vaccine for combating (an infection with) RD-associated Erythroparvovirus in equine, wherein said vaccine is a nucleic acid vaccine comprising a nucleic acid fragment encoding for a CP or NS1 as defined here above, and a pharmaceutically acceptable carrier. As is commonly known, nucleic acid fragments can be used in various ways as active component in a vaccine, for example as DNA in so called DNA vaccination. DNA vaccination is based upon the introduction of a DNA fragment carrying the gene encoding the subunit protein under the control of a suitable promoter, into the host animal. Once the DNA is taken up by the host's cells, the gene encoding the subunit protein is transcribed and the transcript is translated into protein (e.g. CP) in the host's cells. This way, the natural infection process of the parvovirus may be mimicked. Suitable promoters are promoters that are functional in mammalian cells. For example, the expression of the genes can be brought under the control of a heterologous promoter that is functional in a mammalian cell. A heterologous promoter is a promoter that is not the promoter responsible for the transcription of the gene in the wild-type form of the novel equine parvovirus according to the invention. It may be a parvoviral promoter responsible for the transcription of a CP or NS1 of another parvovirus, that does not belong to the parvoviruses according to the invention or it may be a non-parvoviral promoter. A DNA fragment carrying the gene encoding the CP or NS1 under the control of a suitable promoter could e.g. be a plasmid. This plasmid may be in a circular or linear form.

Examples of successful DNA vaccination of horses are i.a. the successful vaccination against Aujeszky's disease as described in Gerdts et al, Journal of General Virology 78: 2139-2146 (1997). They describe a DNA vaccine wherein a DNA fragment is used that carries glycoprotein C under the control of the major immediate early promoter of human cytomegalovirus. Vaccination was done four times with two weeks intervals with an amount of 50 μg of DNA. Vaccinated animals developed serum antibodies that recognised the respective antigen in an immunoblot and that exhibited neutralizing activity.

Another example of successful DNA vaccination of horses is given by Gorres et al., Clinical Vaccine Immunology 18: 1987-1995 (2011). They described successful DNA vaccination of horses against both pandemic and classical swine H1N1 influenza. They vaccinated with a prime vaccination and 2 homologous boosts at 3 and 6 weeks post priming, of a DNA vaccine comprising the HA gene of influenza H1N1 under the control of a functional promoter.

An alternative type of vaccination with an active that comprises a nucleic acid is RNA vaccination, such as for example RNA vaccination using replicon particles (RPs), as described by Lundstrom, 2014, Vaccines, vol. 6, p. 2392-2415. These RPs are virus-like particles but comprise a defective viral genome and typically, a heterologous gene. These replicon particles comprise RNA packaged in particles (i.e., they are encapsidated) such that they are able to enter a target animal host cell and perform one round of viral genome amplification without the ability to form new particles. The replicon particle does not propagate from the infected cell, as it lacks the necessary structural protein-coding sequence(s). As such, they are more similar to wild-type virus than other replicon vaccines such as naked RNA vaccines, or vaccines comprising RNA launched from a DNA plasmid (Hikke, 2017, Anu. Rev. Anim. Biosci. 2017, 5;10.1-10.21). The genome of the RPs indeed typically expresses a heterologous gene encoding an immunoprotective antigen, such as for example the current CP or NS1. Multiple RNA viruses have been used in the production of RP's, such as members of the positive stranded Flaviviridae, Picornaviridae and Arteriviridae, or negative stranded RNA viruses such as Bunyavirus, Paramyxovirus and Rhabdovirus. Most widely used and most extensively studied are Alphavirus RNA replicon particles (Van der Veen et al., 2012, Anim. Health. Res. Rev., vol. 13, p. 1-9; and: Kamrud et al., 2010, J. Gen. Virol., vol. 91, p. 1723-1727), which are therefore preferred for practical reasons. Also, Alphavirus RPs are believed to be somewhat stronger immunopotentiators than other RPs known in the art and based on other viruses such as the bunyavirus. Several Alphavirus species have been used to develop RP vaccines, e.g.: Venezuelan equine encephalitis virus (VEEV) (Pushko et al., 1997, Virology, vol. 239, p. 389-401), Sindbis virus (Bredenbeek et al., 1993, J. of Virol., vol. 67, p. 6439-6446), and Semliki Forest virus (Liljestrom & Garoff, 1991, Biotechnology (NY), vol. 9, p. 1356-1361).

RP vaccines can elicit mucosal and systemic immune responses following immunization of a target animal (Davis et al., 2002, IUBMB Life, vol. 53, p. 209-211). RP vaccines (VEEV based) are also the basis of several USDA-licensed vaccines, which include: Porcine Epidemic Diarrhea Vaccine, RNA (Product Code 19U5.P1), Swine Influenza Vaccine, RNA (Product Code 19A5.D0), Avian Influenza Vaccine, RNA (Product Code 1905.D0), and Prescription Product, RNA Particle (Product Code 9PP0.00). See also Wang et al., 2018, Vaccine, vol. 36, p. 683-690.

What constitutes an “immunogenically effective amount” for a vaccine according to the invention that is based upon a whole parvovirus according to the invention, subunit protein according to the invention, a live recombinant vector or a nucleic acid vaccine according to the invention depends primarily on the desired medical effect (either preventing, ameliorating or curing the infection and/or resulting disease). The term “immunogenically effective amount” as used herein relates to the amount of parvovirus, empty capsid, live recombinant vector or DNA/RNA vaccine that is necessary to induce an immune response in horses to the extent that it decreases the infection or associated pathological effects caused by infection with a wild-type RD-associated equine parvovirus, when compared to the pathological effects caused by infection with a wild-type RD-associated equine parvovirus in non-immunised horses. It is well within the capacity of the skilled person to determine whether a treatment is “immunologically effective”, for instance by administering an experimental challenge infection to vaccinated animals and next determining a target animal's clinical signs of disease, serological parameters or by measuring re-isolation of the pathogen, followed by comparison of these findings with those observed in field-infected horses.

Many ways of administration can be applied, all known in the art. Vaccines according to the invention are preferably administered to the animal via injection (intramuscular, intraperitoneal, subcutaneous, intradermal route), orally, intra-nasally or rectally. The protocol for the administration can be optimised in accordance with standard vaccination practice. Administration through an intradermal injector (e.g. the IDAL® injector as available via MSD Animal Health, Boxmeer, The Netherlands) is a convenient, safe and effective way of administration of a vaccine according to the invention.

The invention is also embodied in a diagnostic test kit for the detection of antibodies reactive with the novel virus or the novel virus itself, wherein the said test kit comprises such a virus and/or the corresponding Capsid Protein and/or the non-structural protein 1, or the test kit comprises antibodies reactive with the said virus, CP or NS1, or the test kit comprises a PCR primer set that is specifically reactive with a region of the Capsid

Protein gene or NS1 gene of the said virus as exemplified below. For efficient protection against disease, a quick and correct detection of the presence of the RD-associated equine parvovirus is important. The tools in the kit rely on the availability of antibodies against the virus. Such antibodies can e.g. be used in diagnostic tests for RD-associated equine parvovirus.

Antibodies or antiserum comprising antibodies against the RD-associated equine parvovirus according to the invention can quickly and easily be obtained through vaccination of e.g. horses, poultry or e.g. rabbits with the virus according to the invention followed, after about four weeks, by bleeding, centrifugation of the coagulated blood and decanting of the sera. Such methods are well-known in the art.

Other methods for the preparation of antibodies raised against the RD-associated equine parvovirus, which may be polyclonal, monospecific or monoclonal (or derivatives thereof) are also well-known in the art. If polyclonal antibodies are desired, techniques for producing and processing polyclonal sera are well-known in the art for decades. Monoclonal antibodies, reactive against the virus according to the invention can be prepared by immunizing inbred mice by techniques also long known in the art.

A diagnostic test kit based upon the detection of a virus according to the invention or antigenic material of that virus and therefore suitable for the detection of RD-associated equine parvovirus infection may e.g. comprise a standard ELISA test. In one example of such a test the walls of the wells of an ELISA plate are coated with antibodies directed against the virus. After incubation with the material to be tested, labeled antibodies reactive with the virus are added to the wells. If the material to be tested would indeed comprise the novel equine parvovirus according to the invention, this virus would bind to the antibodies coated to the wells of the ELISA. Labeled antibodies reactive with the virus that would subsequently be added to the wells would in turn bind to the virus and a color reaction would then reveal the presence of antigenic material of the virus.

The design of the immunoassay may vary. For example, the immunoassay may be based upon competition or direct reaction. Furthermore, protocols may use solid supports or may use cellular material. The detection of the antibody-antigen complex may involve the use of labeled antibodies; the labels may be, for example, enzymes, fluorescent-, chemoluminescent-, radio-active- or dye molecules. Suitable methods for the detection of antibodies reactive with a virus according to the present invention in the sample include, in addition to the ELISA mentioned above, immunofluorescence test (I FT) and Western blot analysis.

An alternative but quick and easy diagnostic test for diagnosing the presence or absence of a virus according to the invention is a PCR test as described here below, comprising a PCR primer set reactive with a specific region of the CP or the NS1 gene of RD-associated equine parvovirus. Specific in this context means unique for e.g. the CP or the NS1 gene of RD-associated equine parvovirus, i.e. not present in other members of the family Parvoviridae. Preferably such a test would use the primer set (SEQ ID NO: 9-14) that specifically reacts with the Capsid Protein of the virus.

It goes without saying, that more primers can be used than the primers identified above. The present invention provides for the first time the unique sequence of the CP and the NS1 gene of RD-associated equine parvovirus. This allows the skilled person to select without any additional efforts, other selective primers. By simple computer-analysis of the CP or the NS1 gene of RD-associated equine parvovirus gene sequence provided by the present invention with the, known, CP or NS1 gene of other, non-RD-associated, equine parvovirus members of the family Parvoviridae, the skilled person is able to develop other specific PCR-primers for diagnostic tests for the detection of a RD-associated equine parvovirus and/or the discrimination between an RD-associated equine parvovirus and other viral (equine) pathogens. PCR-primers that specifically react with the CP or the NS1 gene of RD-associated equine parvovirus are understood to be those primers that react only with the CP or the NS1 gene of RD-associated equine parvovirus and not with the CP or the NS1 gene of another (equine) pathogenic virus, or group of (equine) pathogenic viruses.

### EXAMPLES

Example 1: Detection of a Novel RD-Associated Virus Using VIDISCA

Serum samples and nasal swabs taken from horses at the Index farm (see Example 4 below) were submitted for analysis in a virus discovery platform.

Samples were collected from subjects with clinical symptoms of respiratory distress and analysed for the presence of viruses by VIDISCA (Virus discovery based on cDNA-AFL (amplified fragment length polymorphism), a method originally described by van der Hoek et al., (Nat Med. 2004; 10:368-373). Virus discovery based on VIDISCA is a Next Generation Sequencing-based approach that provides a fast and effective tool for amplification of unknown genomes. The VIDISCA method is based on double restriction enzyme processing of a target sequence and ligation of oligonucleotide adaptors that subsequently serve as priming sites for amplification. As the method is based on the common presence of restriction sites, it results in the generation of reproducible, species-specific amplification patterns.

Using the VIDISCA method, a total of 8 different unique DNA fragments (flanked by Msel sites) were identified in serum samples of affected horses. The fragments showed homology at the translated protein level with Chipmunk Parvovirus as available in the NCBI Genbank under accession numbers YP_009507377.1 (VP1 protein) and NC_038543.1 (nucleotide). See Yoo B C, Lee D H, Park S M, Park J W, Kim C Y, Lee H S, Seo J S, Park K J, Ryu W S. A novel parvovirus isolated from Manchurian chipmunks, Virology, 1999 Jan 20;253(2):250-8 and Chen Z, Chen AY, Cheng F, Qiu J. Chipmunk parvovirus is distinct from members in the genus Erythrovirus of the family Parvoviridae, PLoS One. 2010 Dec. 3;5(12):e15113. doi: 10.1371/journal.pone.0015113.

Via bridging PCRs, using primer design based on the obtained 8 VIDISCA fragments, and BDT Sanger sequencing, a first version of an almost full genome sequence of the novel virus (4.8 Kb) could be obtained (SEQ ID NO: 1).

The sequence of the novel viral genome was analysed and revealed that the novel virus belongs to the genus of the Erythroparvoviruses of the Parvovirinae subfamily within the Parvoviridae. The part of the VP1 coding sequence that was identified in VIDISCA was used in a translated BLAST to search for homologous proteins in the NCBI database. This analysis revealed that for this particular fragment, the level of identity between the novel virus and chipmunk parvovirus YP_009507377.1 (the closest Erythroparvovirus) was a mere 46% at the translated protein level.

Some parts of the sequence need further experimental verification, in particular the extreme 5′ part of the virus genome is missing. As a consequence of that, the start codon for the NS1 protein has not been identified yet. It is estimated that approximately 100 N-terminal amino acids are missing based on alignments with Chipmunk parvovirus

NS1 (NC_038543.1), Bovine parvovirus 3 (AF406967) and Human parvovirus B19 (AY386330).

The ORF2 coding sequence was obtained completely and again BLAST analysis was used to study homology with protein-coding sequences in the database. The data confirmed homology with chipmunk parvovirus VP1. Furthermore, BLAST analysis using the partial NS1 sequence confirmed homology at the protein level with Chipmunk parvovirus and Bovine parvovirus 3.

The phylogenetic tree of the partial NS1 protein is presented in FIG. 1, that of ORF2-encoded VP1 protein in FIG. 2. As follows from these phylogenetic trees, the novel equine parvovirus belongs to the sub-family of Erythroparvoviruses, such as Chipmunk parvovirus and the human parvovirus B19. The NS1 and CP show low-level identity to the characterised homologous proteins in Chipmunk parvovirus and Human parvovirus B19. In Human parvovirus B19, the viral capsid is formed by 60 subunits composed of ˜95% VP2 of 554 amino acids and ˜5% VP1, which is cocarboxyterminal with VP2 but has an N-terminal extension of 227 amino acids. It can be deducted from the presently found sequences that a similar putative N-terminal extension of capsid proteins exists in the RD-associated virus.

### Example 2: Phylogenetic Trees

The phylogenetic tree of the partial NS1 protein is presented in FIG. 1, that of ORF2-encoded VP1 protein in FIG. 2. The percentage bootstrap support is specified at the nodes. Distance bars indicate the number of nucleotide substitutions per site. For construction of the phylogenetic tree, Genbank entries of reference viruses of the different genera were included (based on information of the ICTV, international committee on taxonomy of viruses), as well as the Genbank entries of previously described equine parvoviruses.

The amino acid sequences of the partial NS1 protein and VP1 of the novel virus were used to calculate phylogenetic trees based on the Neighbor-Joining method. The percentage of replicate trees in which the associated taxa clustered together in the bootstrap test (500 replicates) are shown next to the branches in FIGS. 1 and 2. The tree is drawn to scale, with branch lengths in the same units as those of the evolutionary distances used to infer the phylogenetic tree. The evolutionary distances were computed using the Poisson correction method and are in the units of the number of amino acid substitutions per site. This analysis involved 20 amino acid sequences. All ambiguous positions were removed for each sequence pair (pairwise deletion option).

Evolutionary analyses were conducted in MEGA X.

In more detail, regarding FIG. 1, NS1 sequences or partial NS1 sequences of previously identified parvoviruses of different genera (ICTV), as well as all equine parvoviruses, were selected for evolutionary analysis. Accession numbers are mentioned in the Figure. The evolutionary history was inferred using the Neighbor-Joining method (Saitou N. and Nei M, 1987: The neighbor-joining method: A new method for reconstructing phylogenetic trees. Molecular Biology and Evolution 4:406-425). The optimal tree with the sum of branch length=10,74790667 is shown. The percentage of replicate trees in which the associated taxa clustered together in the bootstrap test (500 replicates) are shown next to the branches (Felsenstein J, 1985: Confidence limits on phylogenies: An approach using the bootstrap. Evolution 39:783-791). The tree is drawn to scale, with branch lengths in the same units as those of the evolutionary distances used to infer the phylogenetic tree. The evolutionary distances were computed using the Poisson correction method (Zuckerkand and Pauling (1965). Evolutionary divergence and convergence in proteins. Edited in Evolving Genes and Proteins by V. Bryson and H. J. Vogel, pp. 97-166. Academic Press, New York) and are in the units of the number of amino acid substitutions per site. This analysis involved 20 amino acid sequences. All ambiguous positions were removed for each sequence pair (pairwise deletion option). There were a total of 934 positions in the final dataset. Evolutionary analyses were conducted in MEGA X (Kumar S., Stecher G., Li M., Knyaz C., and Tamura K. (2018). MEGA X: Molecular Evolutionary Genetics Analysis across computing platforms. Molecular Biology and Evolution 35:1547-1549).

Regarding FIG. 2, the same method was used based on VP1 sequences of previously identified parvoviruses of different genera (ICTV), as well as all equine parvoviruses.

### Example 3: PCR and Genome Sequencing of the Novel Virus

A way to establish the presence of the virus according to the invention in a test sample is a quantitative PCR-test using a primer set that is specific for the Capsid Protein gene sequence of this virus. A primer set of which the sequence is depicted in SEQ ID NO: 9-10 was elected for their specificity for the virus. The quantitative PCR-test using the primer set that specifically reacts with the Capsid Protein gene of the virus uses the two primers (SEQ ID NO: 9-10), i.e. RDAV FW2 and RDAV RV2. This primer set can be used in SYBR-green based qPCR analysis such as for example the Biorad SsoAdvanced protocol.

If a member of the of the subfamily of the Parvovirinae subfamily within the Parvoviridae is analysed using the primer sets described above, the following can be said: if an analysis of the PCR-product of the primer set reveals a PCR product of approximately 76 base pairs, and the melting curve corresponds to the melting curve of the quantification control sample in the assay as explained in the Examples section, this demonstrates that a virus according to the invention is present in the sample. Merely as an example: a PCR product of approximately 76 base pairs is a PCR product with a length of between 76+5 and 76−5 base pairs. The dilutions series of the quantification control samples can be used to estimate the viral concentration in the sample, based on linear regression on the standard curve dilution series.

Still another way to establish the presence of the virus according to the invention in a sample depends on a PCR-test using primer sets that are specific for the VP1 Capsid Protein gene sequence a virus according to the invention. Two different primer sets, which together compose a so-called Nested PCR primer set of which the sequence is depicted in SEQ ID NO: 11-14 were elected for their specificity for the virus. The PCR-test using the first primer set (SEQ ID NO: 11+13) that specifically reacts with the Capsid Protein gene of the virus uses the two primers RDAV F and RDAV R. The PCR-test using the second primer set (SEQ ID NO: 12+14) reacts specifically with the capsid protein gene that is amplified by the first primer set (nested PCR), resulting in increased sensitivity of detection of the PCR test. The Nested PCR uses the two primers RDAV Nested F and RDAV Nested R.

If a member of the of the subfamily of the Parvovirinae subfamily within the Parvoviridae is analysed using the primer sets described above, the following can be said: if an analysis of the PCR-product of the first primer set reveals a PCR product of approximately 209 base pairs or if analysis of the PCR-product of the Nested primer set reveals a PCR product of approximately 141 base pairs, this demonstrates that a virus according to the invention is present in the analysed sample. Merely as an example: a PCR product of approximately 209 base pairs is a PCR product with a length of between 209+10 and 209−10 base pairs. A PCR product of approximately 141 base pairs is a PCR product with a length of between 141+10 and 141−10 base pairs.

Nucleotide isolation: PCR and qPCR were performed on extracted serum samples and extracted nasal swab samples. Nucleic Acids were extracted using the Magnapure methodology (Roche).

qPCR RD-associated virus: A quantitation was performed based on a titration series of a pUC57 plasmid containing the amplicon with known concentrations.

Sequence pUC57 positive control plasmid insert: SEQ ID NO: 8.

The positive control plasmid was ordered from Genscript based on SEQ ID NO: 8, which originates from the identified VP1 sequence of RD-associated virus in SEQ ID NO: 1.

Experiments were conducted on Biorad equipment and analysed using Biorad CFX software with reaction conditions as depicted below.

TABLE 1
qPCR reaction RDAV/Sybr green

| | Final | Amount per | |
|---|---|---|---|
| qPCR | conc. (nM) | reaction | |
| WFI | | 6.5 | μl |
| Biorad Sso advanced Sybr green supermix | 1x | 12.5 | μl |
| Foward primer | 200 nM | 0.5 | μl |
| Reverse primer | 200 nM | 0.5 | μl |
| Extracted nucleotide sample | | 5.0 | μl |
| END VOLUME | | 25.0 | μl |

TABLE 2
qPCR primers RDAV/Sybr green

| | | Tm |
|---|---|---|
| qPCR primers | Sequence | (° C.) |
| RDAV FW2 | GACCCGGTATATGAACCCATC | 61 |
| (SEQ ID NO: 9) | | |
| RDAV RV2 | GACGCATGACCATCTGGTAG | 62 |
| (SEQ ID NO: 10) | | |
| Product size: 76 bp | | |

The test was performed using the following cycling program, which included melting curve analysis.

TABLE 3
qPCR cycling program RDAV/Sybr green
with melting curve analysis

| Step | Temperature | Duration | | |
|---|---|---|---|---|
| Pre-denaturation | 95° C. | 3 | min | |
| Denaturation | 94° C. | 5 | sec | 40 |
| Annealing/elongation | 58° C. | 10 | sec | cycles |

| | | |
|---|---|---|
| Melting curve | 65° C.-95° C. | 0.5° C./5 sec |

Nested PCR RD-associated virus:

Performed using Taq polymerase and 55° C. annealing temperature.

| Name primer | Sequence |
|---|---|
| RDAV_F | SEQ ID NO: 11 |
| RDAV_Nested_F | SEQ ID NO: 12 |
| RDAV_R | SEQ ID NO: 13 |
| RDAV_Nested_R | SEQ ID NO: 14 |

1ST PCR: fragment length 209 bp.

Nested PCR: fragment length 141 bp.

### Example 4: Clinical Description and PCR Analysis Farm 1—Index Cases

The Dutch index farm with horses suffering from clinical symptoms as described in relation to the invention was identified. The affected horses were 2-year old and 3-year old.

Abnormal respiratory symptoms in the first affected horse (stallion, 2 years old) with dyspnea were observed on 8 Jan. 2019 in cage 1 in stable 1. Symptoms remained and the horse was placed in isolation on 10 January. Endoscopic analysis took place on January 11, showing paralysis of the larynx. Edema, non-purulent mucus, swollen mucosa and larynx were noted during the endoscopic analysis. A nasal swab was taken on 10 January and 12 January. Also, serum was taken on 12 January. On the 23 January, a second serum sample was taken. Horse eventually died 5 February.

Abnormal respiratory symptoms in the second horse (stallion, 2 years old, housed in the same cage as horse 1) were observed on 12 January. Severe clinical symptoms, dyspnea. Horse was isolated from the group and examined, presence of clotted blood in the nose and difficulties with respiration (pumping') were noted. A nasal swab and serum sample were taken on 12 January. On 23 January, a second serum sample was taken. On 26 February, a third serum sample was taken. On April 10, endoscopic analysis revealed paralysis left, minimal movement right in the larynx, epiglottis weak, bad prognosis.

The sera taken on the 23 January were the sera in which the novel virus RDAV was identified by VIDISCA (see Example 1). An overview of PCR results is presented below in Table 4.

TABLE 4
(q)PCR and VIDISCA results on two
horses diagnosed with clinical symptoms

| | | Days after onset | qPCR | Sq copies | Melting | Nested |
|---|---|---|---|---|---|---|
| Horse | Sample | symptoms | RDAV | per μl | curve | RDAV |
| 1 | swab | 2 | Pos | 8.14E−01 | Specific | Pos |
| 1 | Swab | 4 | Neg | — | — | Pos |
| 1 | Serum | 4 | Neg | — | — | Neg |
| 1 | Serum | 15 | Pos | 5.82E+03 | Specific | Pos |
| 2 | Swab | 0 | Neg | — | — | Neg |
| 2 | Serum | 0 | Neg | — | — | Neg |
| 2 | Serum | 11 | Pos | 1.05E+06 | Specific | Pos |
| 2 | Serum | 45 | Pos | 1.06E+02 | Specific | Pos |

The qPCR is the analysis result of qPCR RDAV (real-time PCR) classified as positive (Pos) or negative (Neg). The Sq is a calculated viral load in copies/μl, the calculation being based on a standard curve. If a value is below 10, the calculated value is outside of the standard curve. A positive sample is then identified based on the presence of an amplicon-specific melting curve. The Nested RDAV is the result of the nested PCR classified as positive (Pos) or negative (Neg).

A total of 31 clinical cases were diagnosed at the farm in the period until June 2019. The cases in cage 1 stable 1 (all stallions) were between 8 January and 8-February (horses born 2017). The case in cage 1 stable 2 (a mare) was on 26 January (horse born 2017). The cases in cage 2 stable 2 (all stallions) were between 9-20 February (horses born 2017). The cases in cage 1 stable 3 (all stallions) were between 11-21 February (horses born 2017). The cases in cage 2 stable 3 (all stallions) were between 11 February-13 March (horses born 2016). The case in cage 2 stable 1 (a stallion) was on 18 Feb (horse born 2017).

The number of clinically affected horses was 31. The number of mortalities was 7, 1 mare, 6 stallions. Three stallions needed to be euthanised. Serum samples of 10 of the 29 additional horses with clinical symptoms were analysed for presence of viral pathogens. The results are shown in Table 5. Horse 6 was the mare. Samples were all serum.

TABLE 5
(q)PCR results on 10 of the 29 additional
horses diagnosed with clinical symptoms

| Stable/ | | Days after onset | qPCR | Sq copies | Melting | Nested |
|---|---|---|---|---|---|---|
| Cage | Horse | symptoms | RDAV | per μl | curve | RDAV |
| 2/1 | 6 | 31 | Pos | 1.65E+02 | Specific | Pos |
| 1/1 | 7 | 18 | Pos | 4.29E+02 | Specific | Pos |
| 2/2 | 8 | 17 | Pos | 2.70E+01 | Specific | Neg |
| 3/1 | 11 | 15 | Pos | 1.56E+01 | Specific | Pos |
| 3/2 | 14 | 15 | Pos | 9.78E+00 | Specific | Pos |
| 2/2 | 15 | 15 | Pos | 1.15E+01 | Specific | Pos |
| 2/2 | 20 | 13 | Pos | 8.19E+01 | Specific | Pos |
| 1/2 | 22 | 8 | Pos | 2.60E+02 | Specific | Pos |
| 3/1 | 23 | 7 | Pos | 1.51E+01 | Specific | Pos |
| 3/2 | 25 | 7 | Neg | — | — | Neg |

As can be seen, the cumulative number of positive cases was 9/10 for the regular qPCR and 8/10 for the nested RDAV PCR. In stable 1 cage 1, two horses without clinical symptoms were sampled as in-cage controls. In stable 3, cage 2, four horses without clinical symptoms were samples as in-cage controls. The results of the PCR analyses are shown in Table 6. The horse were all stallions, all samples were serum samples.

TABLE 6
(q)PCR results of six horses housed in the same
cages but not diagnosed with clinical symptoms

| Stable/ | | qPCR | Sq copies | | |
|---|---|---|---|---|---|
| Cage | Horse | RDAV | per μl | Melting curve | Nested RDAV |
| 1/1 | C1 | Pos | 5.95E+01 | Specific | Pos |
| 1/1 | C2 | Pos | 3.67E+01 | Specific | Pos |
| 3/2 | C3 | Pos | 1.05E+01 | Specific | Pos |
| 3/2 | C4 | Neg | — | — | Neg |
| 3/2 | C5 | Neg | — | — | Neg |
| 3/2 | C6 | Pos | 1.2E+01 | Specific | Pos |

Of the horses housed in the same cage, with direct contact, but without clinical symptoms, 4/6 tested positive on the novel virus.

### Control Samples

A total of 55 serum samples of horses (1-year-old) originating from multiple locations different from the index farm and without clinical symptoms of respiratory distress were tested in the qPCR for presence of the novel virus according to the invention. 46/55 samples tested negative for the novel virus. 7/55 samples tested positive based on melting curve, but with a viral load <10\hat{ }1 copies/μl and 2/5 samples tested positive with a quantifiable viral load of 1.78×10\hat{ }1 copies/μl and 3.05×10\hat{ }1 copies/μl respectively.

### Example 5: Clinical Description and PCR Analysis Farm 2

A second farm with horses suffering from clinical symptoms as described in relation to the invention was identified in retrospect based on clinical symptoms reported. Clinical problems at the farm started in February-March 2019.

About 20 horses developed clinical symptoms, and 2 horses died. Clinical symptoms included sudden onset of respiratory distress and in two cases sudden death with epistaxis (left and right) after excitation. On laryngeal examination total or hemi-laryngeal paralysis. Later, about 60% of the affected horses developed a form of ataxia (grade 1/5-3/5). It seemed that the horses were slowly getting better in time. The affected horses were two-year-olds and three-year-olds.

One horse that was affected had left the stud farm for a month without any clinical signs and thereafter got the laryngeal paralysis on the new location and died.

In April, endoscopic analysis was performed on all horses on the farm, and about 40 out of 60 stallions showed a unilateral paralysis of the larynx (hemiplegia larynges) of grade 1-2 (complete paralysis=grade 4). All bilateral paralysis cases had died. In the mares, 16 out of 20 showed some grade of hemiplegia larynges.

Serum samples and nasal swabs were taken from five clinically affected horses in July, 4-5 months after the start of clinical symptoms. The qPCR analysis for presence of the novel equine parvovirus showed amplification above detection level (presence of amplicon-specific melting curve) in 4 out of 5 serum samples. All nasal samples tested negative for the novel virus.

### Example 6: Clinical Description and PCR Analysis Farm 3

A third farm with horses suffering from clinical symptoms as described in relation to the invention was identified. The horses were two-year olds. Clinical symptoms were first observed on November 11, the date of sampling was December 18. The number of mortalities was 1 and the number of clinically affected horses was 4. The percentage of mortalities based on total number of clinically affected horses was 25%. The results are summarised in Table 7. All horses were mares. Horses 1-4 were the clinically affected horses, of which horse 1 did die before samples were taken. The other three affected horses shoed bilateral paralysis symptoms. Of the three clinically healthy horse, horse 6 also showed bilateral paralysis. The other showed unilateral paralysis

TABLE 7
qPCR results and clinical observations Farm 3

| | qPCR | Sq copies | Melting | qPCR | Melting |
|---|---|---|---|---|---|
| | RDAV | per μl | curve | RDAV | curve |
| Horse | serum | serum | serum | swab | swab |
| 1 | — | — | — | — | — |
| 2 | Pos | 4.28E+01 | Specific | Pos | Specific |
| 3 | Pos | 1.98E+01 | Specific | Pos | Specific |
| 4 | Pos | 7.75E+00 | Specific | Neg | — |
| 5 | Pos | 1.34E+01 | Specific | Pos | Specific |
| 6 | Pos | 9.94E+01 | Specific | Pos | Specific |
| 7 | Pos | 3.31E+01 | Specific | Pos | Specific |

### Example 7: Culture of the Virus

Successful in vitro culture of the novel virus requires that a primary or permanent culture (novel or existing cell line) of permissive cells can be established. Alternatively, a reverse genetics system can be established based on the genomic sequence of the novel virus.

A permissive cell is defined as the cell that expresses the appropriate cellular receptors to bind and internalize a viral pathogen, replicates the viral genome, and produces infectious virus. The likely mechanism for viral entry, the molecular basis of cell permissiveness, genome replication and virion assembly can be deducted from viruses that have a relatively close genetic relationship to the novel virus, for example human parvovirus B19. The knowledge on B19V infection models in in vitro cultures thus serves as a starting point and template to study cellular requirements for the novel virus (trophism and ssDNA replication, establishment of an infectious clone).

B19V is known to have tropism for human erythroid progenitor cells (hEPCs). This trophism is related to the presence of a globoside (Gb4Cer) receptor, which is also present in some non-erythroid tissues such as endothelial cells, fetal hepatocytes, placental trophoblastic cells and some megakaryocytes cells (Kishore et al., Indian J Med Res. 2018 Oct; 148(4): 373-384). Globoside is also known as blood group P antigen (Brown et al., SCIENCE, Vol. 262, 1 OCTOBER 1993, p.114). Human bone marrow cells that lack globoside on the cell surface are resistant to B19 infection (Brown et al., N Engl J Med 1994 330 1192-6).

Globoside on the membrane of erythroid progenitor cells binds to the VP2 protein of B19V. The tissue distribution of globoside (von dem Borne et al., British Journal of Hematology. 1986, 63, 35-46) correlates with the tropism of B19V and its presence on the cell surface is thus a major determinant of viral tropism. More specifically, globoside is dispensable for B19V entry but essential at a postentry step for productive infection (Bieri and Ros, 2019. Journal of Virology, Volume 93 Issue 20 e00972-19). Intracellular factors, possibly specific for the erythroid lineage, are also required for complete expression of viral genome and for viral infectivity (Liu et al., Journal of Virology, Vol. 66, No. 8 Aug. 1992, p. 4686-4692).

It follows that the tissue trophism of the novel virus can be determined after the binding partner of the novel virus VP2 has been identified. As the VP2 protein sequence of the novel virus is now known, it is possible to use for example the methodology used by Wang et al., Virology 490 (2016) 59-68, who used a viral capsid protein as a bait in the Yeast-2-Hybrid system to identify the binding partner of the viral capsid protein. Moerdyk-Schauwecker et al., Virol Methods. 2011 May; 173(2): 203-212, described a modification of the classical Yeast-2-Hybrid system to study protein-protein interactions of another virus, VSV. Once such binding partner, a cellular receptor protein, is identified, it is possible to use publicly available databases such as NCBI to identify the cell-type and tissue expression pattern of such a receptor in horses or other species. Primary cultures of cells or cell lines can subsequently be used to establish an in vitro culture.

Cell lines that are permissive for B19V are for example UT7/Epo (Shimomura et al., Blood, Vol 79, No 1 (January I), 1992: pp 18-24), KU812Ep6 (Miyagawa et al., Journal of Virological Methods 83 (1999) 45-54), JK-1 (Takahashi et al., Arch Virol (1993) 131:201-208), MB-02 (Munshi et al., Journal of Virology, Vol. 67, No. 1 Jan. 1993, p. 562-566), and UT7/Epo-S1 (Morita et al., Journal of Virology, Vol. 75, No. 16 Aug. 2001, p. 7555-7563). All cells express the Globoside receptor.

B19V possess α5β1 integrin and Ku80 autoantigen as possible co-receptors. Ku80 is a nuclear protein present in immune cells, erythroblasts, B-cells, T-cells, macrophages in bone marrow, tonsils and follicular dendritic cells in the joints. Ku80 autoantigen might assist in virion attachment, and α5β1 integrin in internalization (reviewed by Luo and Qiu, Future Virol. (2015) 10(2), 155-167). However, cell surface expression of Ku80 was shown to be very low in ex vivo-expanded CD36+ hEPCs (less than 5% positive) and other B19V-permissive cells (Luo and Qiu, Future Virol. (2015) 10(2), 155-167), which indicates that the presence of this co-receptor may not be essential for virus entry.

Furthermore, the N-terminal part of VP1 (N-terminal extension of VP2 because of alternative ATG use) can be used as a bait in a Y2H as described above to identify possible additional cellular interacting proteins that play a role in attachment and internalization. N-terminal amino acids 5-80 of VP1 of B19V contain neutralizing epitopes and mediate endosomal uptake.

Human parvovirus B19 virus (B19V) requires dividing cells in order to replicate, as it uses the host polymerase for DNA replication. Generally, DNA replication of autonomous parvoviruses happens in the S phase of the host cell cycle and follows a ‘rolling hairpin’ model of DNA replication. The B19V genome is flanked by two identical inverted terminal repeats (ITRs) that form an imperfect palindrome at each end, a feature also shared by adeno-associated virus 2 (AAV2) and human parvovirus 4. By contrast, all other members of the Parvovirinae subfamily have asymmetric terminal repeats. The ITRs are essential for viral genome replication (hairpin-primed ssDNA replication model) (Luo and Qiu, Future Virol. (2015) 10(2), 155-167).

Once an in vitro culture method for the novel virus has been established, or if horse tissue is obtained in which the virus actively replicates, it is possible to construct an infectious clone following the methodology of Zhi, N., Z. Zadori, K. E. Brown, and P. Tijssen. 2004. Construction and sequencing of an infectious clone of the human parvovirus B19. Virology 318:142-152. Such infectious clone can also be generated once the complete sequence of the novel virus has been obtained, for example based on PCR and sequencing of infected horse tissues wherein the virus actively replicates. It is essential to identify the correct ITRs of the novel virus as these are critical for replication of the virus. However, a 67-nucleotide region of the B19V minimum DNA replication origin has been identified and a hairpin-independent model of B19V DNA replication has been proposed by Guan et al., J. Virol. 83: 9541-9553 (2009).

Importantly, the genome of human parvovirus B19, obtained as described above, can replicate in nonpermissive cells (i.e. cell that do not express the viral receptor for VP2 protein) with the help of adenovirus genes, and subsequently produce infectious virus (Guan et al., J. Virol. 83: 9541-9553 (2009).). The failure of B19V DNA replication in nonpermissive human HEK 293 cells can be overcome by expression of the adenovirus E2a, E4orf6, and VA RNA genes by means of transfection of a helper plasmid. Thus, with a complete genome sequence available, a culture system can be established even if the cellular receptor has not been identified.

### Example 8: Erythroparvovirus Diagnostics

Laboratory diagnosis of acute B19V infections are well developed and believed to be correspondingly applicable also for the present erythroparvovirus due to the homology and close relationship. Such diagnosis is usually made by detecting specific immunoglobulin G or IgM antibodies in the serum by ELISA and/or DNA in serum or organ material (depending on type of cell infected, for B19V this is bone marrow). In infected tissues, the parvovirus can be detected by in situ hybridization but more commonly by polymerase chain reaction (PCR) or real-time PCR (qPCR). Also, electron microscopy can be used to demonstrate virions in serum.

An ELISA test for B19V was developed using cloned, baculovirus expressed and purified B19V VP1 and VP2 proteins as antigens. Seroepidemiology and B19V susceptibility of general population to acquire B19V infection can be determined by estimating B19V-specific IgG antibodies to B19V capsid proteins VP1 and VP2 by ELISA.

Also, immunofluorescence tests can be developed for seroepidemiological analysis, for example by expressing VP1 and VP2 in VERO cells.

Antibodies can be generated by injecting the baculovirus/insect cell expressed protein, or E.coli expressed protein, in for example rabbits and mice.

VLP formation of B19V VP2 produced in E.coli has been described by Sanchez-Rodriguez et al., Biochimie 94 (2012) 870e878.

### Example 9: Expression of RDAV VP2 and RDAV VP2-Myc in VERO Cells for Use in Serology Test

For expression studies, the VP2 gene of RDAV (SEQ ID: NO 5) was cloned in a pcDNA3.1 plasmid expression vector (ThermoFisher). The VP2 gene was also cloned in the pcDNA3.1 plasmid expression vector with addition of the coding sequence of a C-terminal Myc-tag (EQKLISEEDL) for immunological detection of the expressed protein. Proteins expressed from these vectors were RDAV VP2 (SEQ ID: NO 15) and RDAV VP2-Myc (SEQ ID NO: 16). Maxiprep plasmid DNA was prepared and diluted to a final concentration of 1 mg/mL.

VERO cells (African Green Monkey) were cultured in tissue culture medium (50%/50% mixture of Earle's Medium Essential Medium (MEM) and Glasgow's modification of MEM (GMEM)) supplemented with 10% Fetal Bovine Serum (FBS). Prior to transfection, microtiter plates (48 wells) were seeded at a density of 3×10⁴ cells/cm² and incubated at 37 C for 24 hours in a humidified CO₂ incubator before transfection.

Transfection mixes were prepared with the Fugene 6 transfection reagent (Promega) according to instructions of the manufacturer. Per 75 μl plain culture medium (see above, no FBS or other additions), 12 μl Fugene 6 transfection reagent was added and allowed to incubate for 5-15 minutes before addition of 2 μl plasmid at a concentration of 1 mg / mL, making a total of 89 μl Fugene-pDNA reagens. This mixture was mixed gently and applied to cells, for example 30 μL was added to a well of a 48-well-plate.

Cells were incubated for an additional 48 hours, after which cells were fixated by replacing 50% of the culture medium in the well for an equal volume of 4% (w/v) Paraformaldehyde (PFA) in PBS (phosphate-buffered saline solution). The cells were fixated at room temperature for 45 minutes in a final concentration of 2% PFA. Subsequently, wells with fixated cells were washed with PBS (3 times) and incubate with 0.25% Triton X-100 in PBS for 5 minutes. Again, wells were washed (3 times) with PBS before staining with primary antibody.

20

The following antibodies/sera were used:

anti-Myc antibody 9E10 directly conjugated to ALEXA-555 (Sigma Aldrich) at the dilution recommended by the manufacturer (1:100 in PBS).

a serum from a horse with no clinical symptoms of respiratory distress and negative in qPCR for RDAV. The serum was titrated to determine the serum dilution at which no non-specific background was observed in the assay. A 1:80 dilution in PBS or higher dilutions gave no non-specific background in the assay.

a serum from a horse that suffered from respiratory distress that was shown to be positive in qPCR for RDAV. The horse was sampled twice (paired serum) and both sera were positive in the qPCR. The second serum sample was used for serology, at a dilution of 1:80 in PBS.

Wells were incubated for 60 minutes 37 C with the antibody/serum. Subsequently, wells were washed (3 times) with PBS and incubated with secondary antibody Goat anti Horse-IgG-ALEXA-488 for detection of RDAV VP2 antibody binding (60 minutes 37 C). Wells were again washed (3 times) with PBS and antibody staining was studied immediately using an UV microscope after addition of mounting medium. For long-term storage, plates were stored under glycerol.

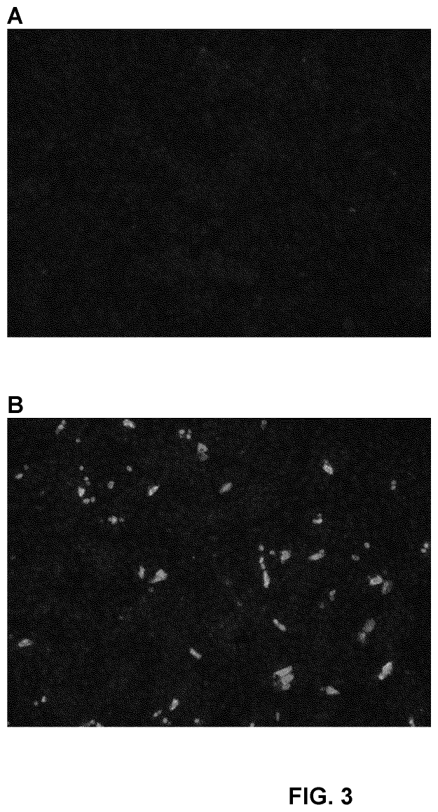

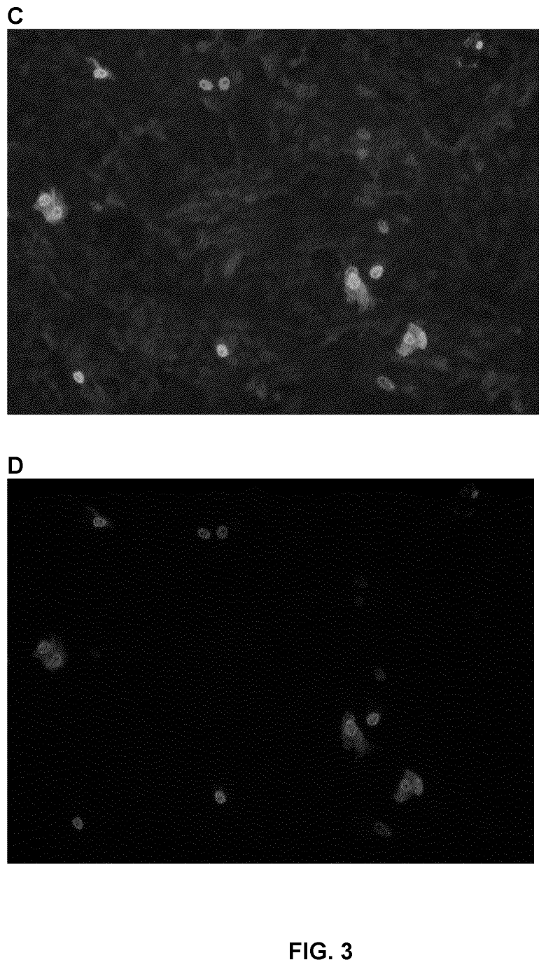

TABLE 8
Results of the RDAV VP serological test validation

| | RDAV VP2 | RDAV VP2-Myc |
|---|---|---|
| Control (no serum/antibody) | negative | negative |
| Horse serum (−) | negative | negative |
| | (FIG. 3A) | |
| Horse serum (+) | positive | positive |
| | (FIG. 3B) | (FIG. 3C) |
| α-Myc 9E10 | negative | positive |
| | | (FIG. 3D) |

The results of the test are presented in Table 8. The Table gives an overview of the combinations of antigen expressed in the VERO cells and antibody/serum used. Per combination, the result on the immunofluorescence test is indicated. Table 8 also refers to microscopic images of the IFTs, shown in FIG. 3 panels A-D.

FIG. 3A shows the (−) horse serum incubated on VERO cells expressing RDAV VP2/Alexa 488 signal. No positive signal is observed, showing absence of RDAV VP2 antibodies in the (−) horse serum (40× magnification)

FIG. 3B shows the (+) horse serum incubated on VERO cells expressing RDAV VP2/Alexa 488 signal. Positive signal reveals the presence of anti-RDAV VP2 antibodies in the (+) horse serum (40× magnification).

FIG. 3C shows the (+) horse serum incubated on VERO cells expressing RDAV VP2-myc (same microscopic field as panel D)/Alexa 488 signal. Positive signal reveals the presence of anti-RDAV VP2 antibodies in the (+) horse serum (100× magnification).

FIG. 3D shows α-Myc 9E10 antibody incubated on VERO cells expressing RDAV VP2-myc (same microscopic field as panel C)/Alexa 555 signal. Positive anti-Myc signal is detected in those cells that are also positive for RDAV VP2. The overlay of panels C and D reveals 100% overlap in signal, which indicates that the (+) horse serum detects 100% of the RDAV VP2-myc transfected cells (100× magnification).

FIGS. 3A and 3B in combination show that only the serum from the infected horse contains antibodies that detect RDAV VP2 expressed in VERO cells. FIG. 3C (Alexa 488 channel, detection of RDAV VP2 antibodies) and FIG. 3D (Alexa 555 channel, anti-Myc; note that C and D are taken from the same microscopic field) together show 100% specificity of the assay. All cells positive in the Alexa 488 channel (antibodies to RDAV VP2) are also positive for the Myc tag. This indicates specificity of the assay. The conclusion of this experiment is that the immunofluorescence test based on plasmid-based expression of RDAV VP2 or RDAV VP2-Myc in VERO cells allows specific detection of antibodies to RDAV VP2 in horse serum, and can thus be used as a serological assay.

### Example 10: Experimental Infection of RDAV Positive Serum taken from Horses with Sudden Onset Severe Respiratory Distress in Ponies

### Outline of the Study

The aim the experiment was to determine if a RDAV-positive serum sample obtained from two horses with sudden onset severe respiratory distress indeed contained infectious virus. Five Shetland ponies (stallions, about 1.5 years of age) were selected for an experimental infection study in which a pooled serum sample from Farm 1 Index case (see Example 4/Table 4) was tested for infectivity. Prior to the study, the ponies were tested for presence of the novel pathogen RDAV in serum by qPCR (according to Example 3) and immunofluorescence based serology (according to Example 9). All five ponies tested negative in both serum qPCR and serology (IgG), indicating that they were not previously infected with RDAV and thus were immunologically naïve to the virus.

Ponies 1-2-3 were housed together in Stable 1 and Ponies 4-5 were housed together in Stable 2. The stables were physically separated from each other, without direct and indirect contact. Pony 1 was experimentally infected by injection of 3 mL inoculum (pooled serum, see Example 4, Table 4, mix of horse 2 day 11 serum and horse 1 day 15 serum) via the intramuscular route. Ponies 2 and 3 served as contact sentinels. Ponies 4 and 5 were kept as negative controls (“NC”; no exposure to RDAV).

Starting from the day of infection, blood samples, rectal swabs, ocular swabs and nasal swabs were collected from all 5 ponies.

Both serum (clot separator blood sample) and plasma (EDTA blood sample) were tested in the lab for presence of RDAV by qPCR analysis as described in Example 4. Blood cell composition (EDTA blood sample) was analyzed using the IDEXX Procyte Dx hematological analyzer within 4 hours after collection according to the manufacturer's instructions. Swabs were collected in Sigma Virocult medium (1 mL, except for nasal swabs 4 mL). Serum, plasma and swab sample were stored at −70 C until tests for presence of the virus were performed by qPCR.

Stable 1 (ponies 1-2-3, group 1) were monitored until day 30 after experimental infection, necropsy was performed on day 35. The ponies were sampled on day 0, 2, 4, 7, 9, 11, 16, 23, and 30 of the study. Stable 2 (ponies 4-5, group 2) were monitored until day 28 of the study, necropsy was performed on day 29 of the study. The ponies were sampled on day 0, 7, 16, 23, and 28 of the study. Prior to necropsy, the ponies were endoscopically examined to detect possible laryngeal dysfunction. At the time of necropsy, organs were macroscopically examined and organ samples were taken for microscopic analysis. Tissue samples were fixed in formalin for this purpose. Samples were processed into H&E stained tissue sections according to the standard procedures described in literature.

### Results

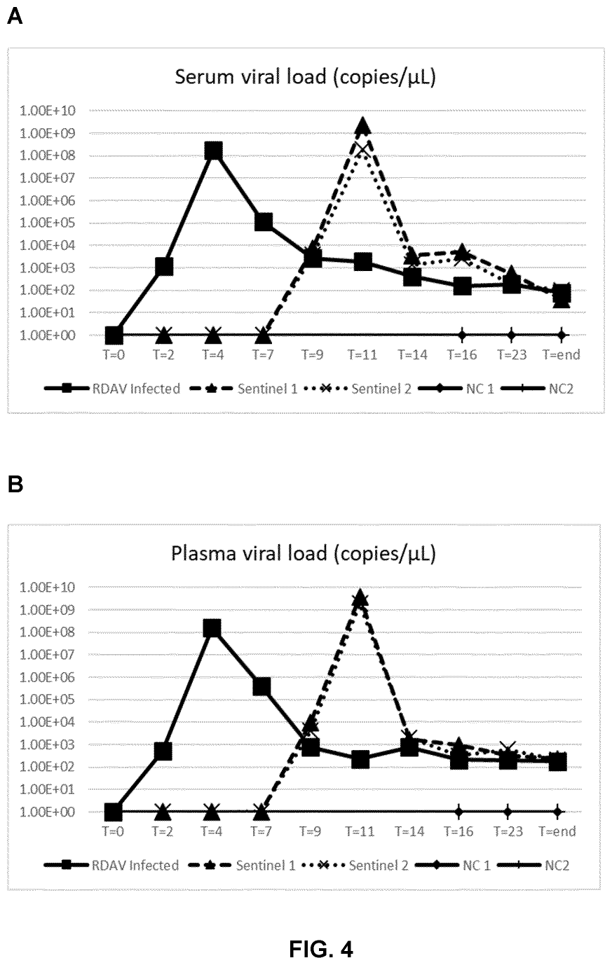

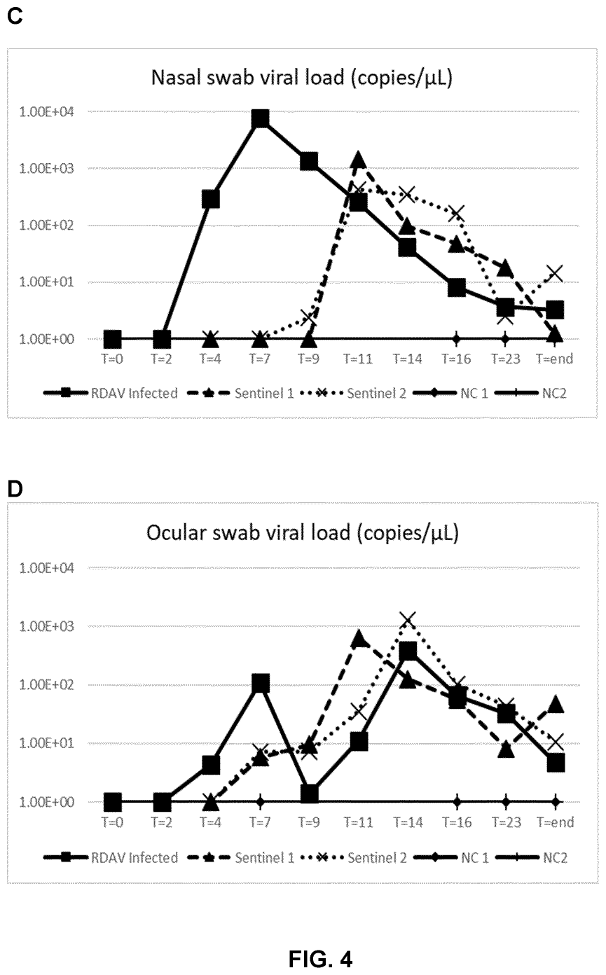

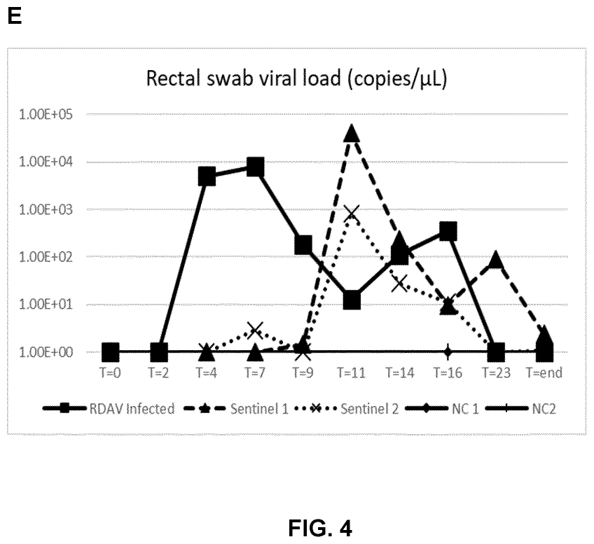

None of the ponies presented with acute respiratory distress during the time course of the study. FIG. 4 panels A-E show the results of the qPCR analyses of serum samples (panel A), plasma samples (panel B), nasal swabs (panel C), ocular swabs (panel D) and rectal swabs (panel E) over time (in days; endpoint T=30). Infection with the RDAV positive pooled serum sample resulted in a clear viremia in blood (serum, plasma, shown by qPCR) in the experimentally infected pony, with a peak around day 4 post inoculation. Also all swabs taken from this pony turned RDAV qPCR positive on day 4 after experimental infection. The viral infection spread to the contact sentinel ponies in the same stable, which subsequently showed peak viremia about 7 days after peak viremia of the experimentally infected pony. Also all swabs taken from the contact sentinels showed a clear positive qPCR at this time. This shows that the inoculum consisting of a pooled serum obtained from two horses with acute respiratory distress contained infectious RDAV, and the virus can now be further isolated from serum using biochemical and ultracentrifugation techniques such as described by Wong et al., 1985 [F. C. Wong, J. G. Spearman, M. A. Smolenski and P. C. Loewen, Equine Parvovirus: Initial Isolation and Partial Characterization. Can J Comp Med 1985; 49: 50-54]. The negative controls remained negative in all qPCRs at all time points.

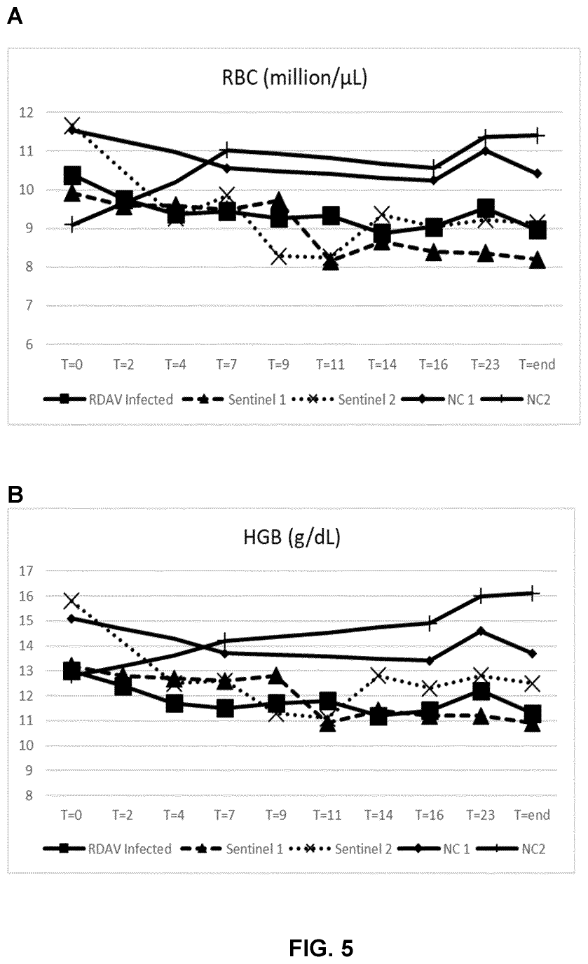

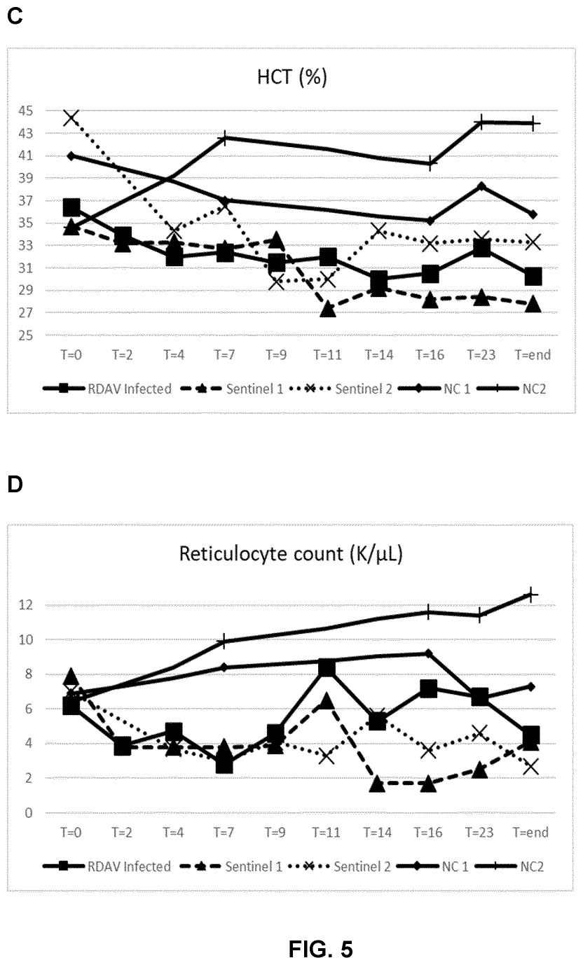

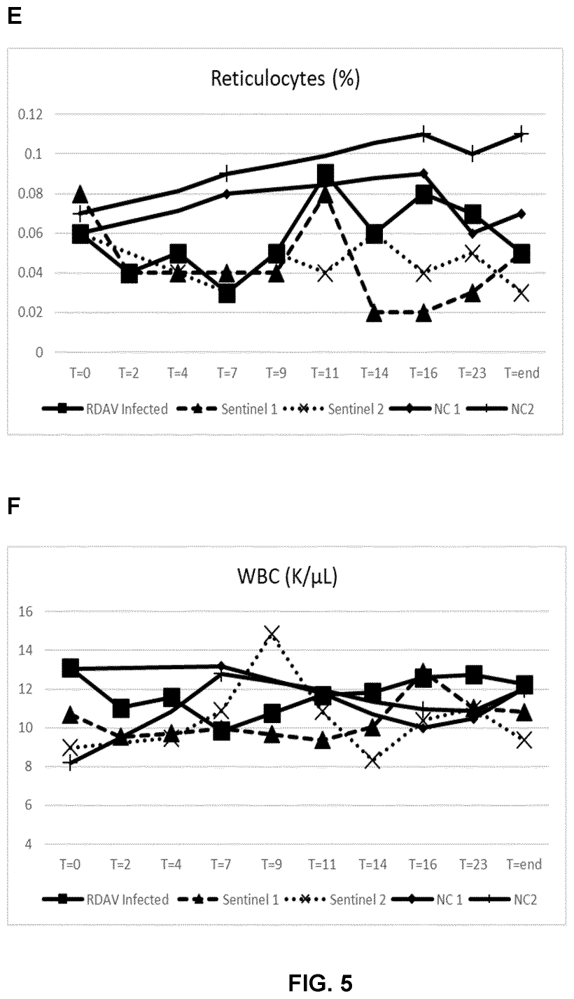

It was investigated whether infection with the novel virus RDAV affected the red blood cell composition to similar extends such as those described for human parvovirus B19. A reference for viremia, clinical features and hematological changes following human parvovirus B19 infection was given by Heegaard and Brown, 2002 [E. D. Heegaard1 and K. E. Brown, Human Parvovirus B19. Clinical Microbiology Reviews, 2002, Vol. 15, No. 3 p. 485-505]. These authors reported a reduction of circulating reticulocytes in B19 patients. Horses differ significantly from humans with regard to circulating red blood cells, specifically, the level of circulating reticulocytes is low. In humans, this percentage varies between 0.5 and 1.5%, whereas in horses this percentage is around 0.1%. Also, a large population of red blood cells is retained in the spleen and not part of active circulation. As in FIG. 4, FIG. 5 shows the results of analysis of Red blood cells (RBC count, panel A), Hemoglobin (HGB, panel B), Hematocrit (HCT, panel C), Reticulocyte count (panel D), Reticulocyte percentage (panel E) and White blood cells count (panel F). An effect of RDAV infection on RBC count, HGB, HCT, reticulocyte count and reticulocyte percentage was noticed (all decreased). However, the white blood cell count was not affected. The permissive cell type for replication of RDAV may therefore reside in the red blood cell lineage, but the red blood cell pattern observed after RDAV infection in horses is not identical to the pattern observed after infection of human B19 virus in humans.

The endoscopic analysis of laryngeal function at the end of the experiment did not reveal abnormalities of vocal cord activity or laryngeal function in any of the ponies.

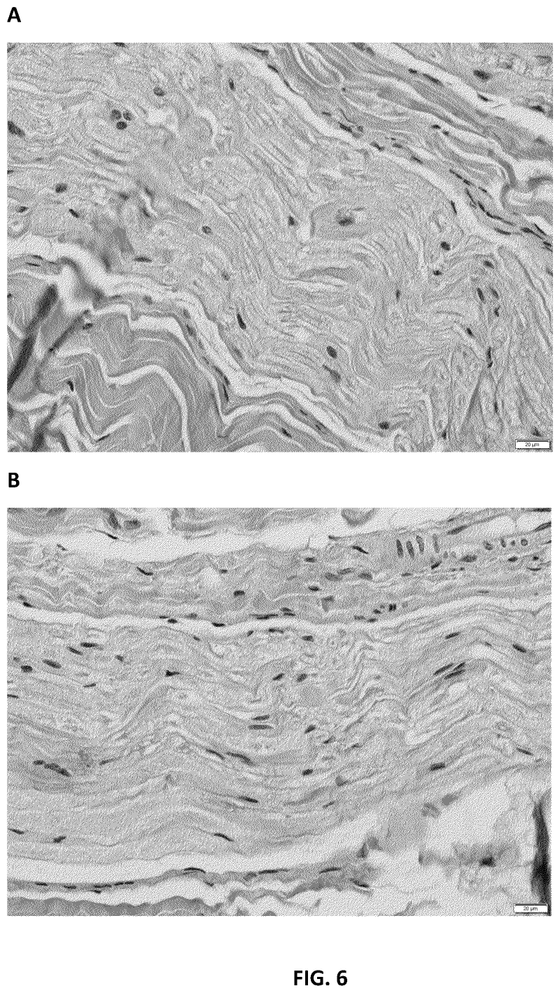

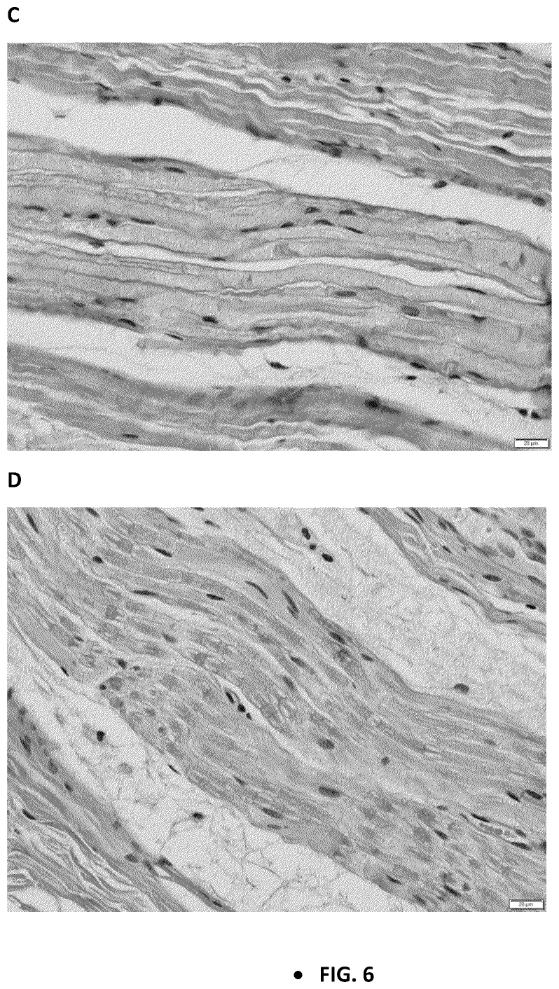

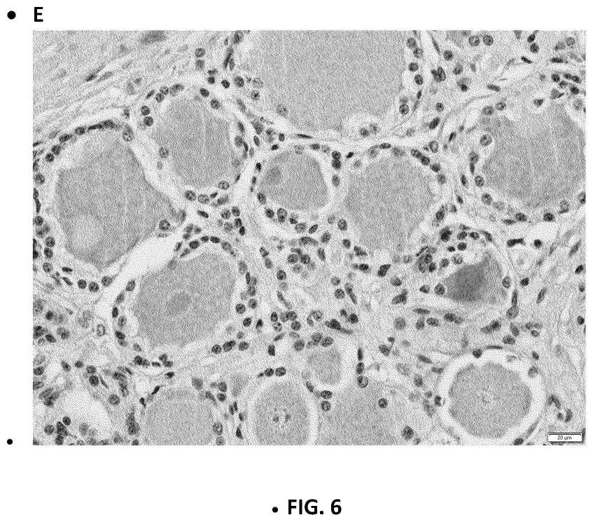

Microscopic analysis of the N. laryngeus recurrens L and R was performed on tissue sections after formalin fixation, paraffin embedding and subsequent H&E stain. The results of this analysis are summarized in Table 9. There are indications for axonal degeneration such as swollen axon material and loss of myelin sheath structures specifically in the experimentally inoculated pony and in one of the two contact sentinels, but not in the controls. This observation links infection with the novel virus RDAV to damage in the nerves that innervate the larynx. Representative pictures of the microscopic sections taken from the N. Laryngeus recurrens (Right) of the RDAV experimentally infected (Panel A) and contact sentinel pony 1 (Panels B,C, NLR Left and Right, respectively) are presented in FIG. 6 (Hematoxylin & Eosin stain of formalin-fixed, paraffin-embedded tissue; scale bar in lower right hand corner 20 μm). Compared to a similar section of a control pony (Panel D, NLR Left), abnormal structures indicative of neuronal damage are visible. Nodes of Ranvier are not clearly recognizable, axonal structures are absent or appear abnormal and myelin sheaths are disrupted. (see Table 9 for further description). However, no infiltrations of immune cells are visible. Also histological sections of the ganglion spinalis at C3 showed abnormal neuronal cell bodies, with nuclear margination, loss of staining, chromatolysis and shrinkage with condensed cytoplasm in the experimentally RDAV infected pony (Panel E).

Based on these data, infection with RDAV can be directly linked to neuronal damage in the innervating nerves of the larynx. The neuronal damage did not clinically translate into laryngeal dysfunction, which may be related to the extent of damage that the 15 neurons had undergone at the time of necropsy. Many studies have identified N.

Laryngeus recurrens-associated neuropathological changes (left more than right) in clinically unaffected horses but mechanisms that lead to the varying severity of laryngeal paralysis from subclinical to severe are largely unknown (Draper and Piercy, 2018) [A. C. E. Draper and R. J. Piercy, Pathological classification of equine recurrent laryngeal neuropathy. J Vet Intern Med. 2018;32:1397-1409]. The current results however show that it is plausible that the novel virus is involved in the occurrence of neuropathological changes in the N. laryngeus recurrens and thus in cases of laryngeal paralysis varying from subclinical to severe. This means that instead of characterizing the novel virus as “associcated with RD distress in equine”, it can also be characterized as being “associated with equine recurrent laryngeal neuropathy”. The clinical presentation may only be present in those cases where severe neuronal damage develops with a large majority of axons affected, caused by RDAV infection only or in combination with other contributing factors that have been associated with laryngeal paralysis. Nonetheless, the knowledge of the novel virus and its association with equine RD and equine recurrent laryngeal neuropathy, may be used to devise vaccines for combatting the corresponding disorders, i.e. equine RD (RD associated with the novel virus) and equine recurrent laryngeal neuropathy.

TABLE 9
Overview of histology results N. laryngeus recurrens

| Animal | Tissue | histology |
|---|---|---|
| RDAV | N. laryng. | Multifocally indications for axonal degeneration with pale |
| Infected | recurrens R | blueish staining, swollen axon material and loss of myelin |
| | | sheath structures. Nerve fascicles surrounded by pale |
| | | basophilic staining material and/or vacuolated cells, |
| | | probably Schwann cells. |
| RDAV | N. laryng. | Minimal nerve tissue present in the slide. |
| Infected | recurrens L | |
| Sentinel 1 | N. laryng. | Multifocally indications for axonal degeneration with pale |
| | recurrens R | blueish staining, swollen axon material and loss of myelin |
| | | sheath structures. Nerve fascicles surrounded by pale |
| | | basophilic staining material and/or vacuolated cells, |
| | | probably Schwann cells. |
| Sentinel 1 | N. laryng. | Multifocally indications for axonal degeneration with pale |
| | recurrens L | blueish staining, swollen axon material and loss of myelin |
| | | sheath structures. Nerve fascicles surrounded by pale |
| | | basophilic staining material and/or vacuolated cells, |
| | | probably Schwann cells. |
| Sentinel 2 | N. laryng. | Nerve fascicles surrounded by pale basophilic staining |
| | recurrens R | material and/or vacuolated cells, probably Schwann cells. |
| Sentinel 2 | N. laryng. | Nerve fascicles surrounded by pale basophilic staining |
| | recurrens L | material and/or vacuolated cells, probably Schwann cells. |
| NC1 | N. laryng. | Minimal nerve tissue present in the slide. |
| | recurrens R | |
| NC1 | N. laryng. | Nerve fascicles surrounded by pale basophilic staining |
| | recurrens L | material and/or vacuolated cells, probably Schwann cells. |
| NC2 | N. laryng. | Nerve fascicles surrounded by pale basophilic staining |
| | recurrens R | material and/or vacuolated cells, probably Schwann cells. |
| NC2 | N. laryng. | Nerve fascicles surrounded by pale basophilic staining |
| | recurrens L | material and/or vacuolated cells, probably Schwann cells. |
| | | Multifocally minimal perivascular mononuclear cell |
| | | aggregation in adjacent connective tissues. |

### Example 11: Culture of RDAV Virus on Cells Isolated from Peripheral Blood

Example 10 shows that (progenitor) cells of the red blood cell lineage are affected by infection with RDAV. It follows that virus culture can be established on cultures of hematopoietic progenitor cells cultured from normal peripheral blood. Such method has been described by [T. F. Schwarz, S. Serke, B. Hottentrager, A. von Brunn, H. Baurmann, A. Kirsch, W. Stolz, D. Huhn, F. Deinhardt, and M. Roggendorf, Hematopoietic Progenitor Cells Generated In Vitro from Normal Human Peripheral Blood. Journal of Virology, February 1992, p. 1273-1276]. Hematopoietic progenitor cells are isolated from heparinized peripheral blood and cultured in the presence of Interleukin 3 (IL-3) and erythropoietin (EPO) after removal of CD3+ cells (CD3 or cluster of differentiation protein 3 is the T-cell coreceptor/T-cell marker) and CD14+ cells (CD14 or cluster of differentiation protein 14 is a marker for macrophages) from the population, for example via antibody labeling followed by removal via magnetic bead-based cell sorting. After culturing the cells for about 12 days in the presence of IL3 and EPO, cells of the erythroid lineage have proliferated, and those cells can subsequently be infected by RDAV. To this end, cells can be diluted to 3×10⁶ cells per mL in RPMI cell culture medium, and RDAV can be added to the suspension in different concentrations, based on copy number or calculated infective dose. The mix can be incubated at 4 C for 4 hours, and thereafter the cells can be washed to remove non-adsorbent virus. The cells can be grown, and replication of the internalized virus can be shown by, for example, qPCR, immunofluorescence or electron microscopy at different time points after infection. For final proof of infectivity of the in vitro cultured virus, harvested virus can be used as inoculum to infect fresh cultures prepared as described in this example.

1. An isolated virus which is a member of the sub-family Parvovirinae of the family of the Parvoviridae, said virus being characterised in that:
the virus is an Erythroparvovirus,
the virus is associated with respiratory distress (RD) in equine and
the virus has a viral genome comprising a nucleotide sequence which has a level of identity of at least 70% to the nucleotide sequence as depicted in SEQ ID NO: 1.

2. A cell culture comprising a virus according to claim 1.

3. A nucleic acid fragment comprising a gene encoding the CP of claim 5, wherein the said gene has a nucleotide sequence that has a level of identity of at least 70% to the nucleotide sequence of the CP gene as depicted in SEQ ID NO: 2.

4. (canceled)

5. A capsid protein (CP) comprising an amino acid sequence which has a level of identity of at least 70% to the amino acid sequence according to SEQ ID NO: 3.

6. A nucleic acid fragment comprising a gene encoding the NS1 claim 8, wherein the said gene has a nucleotide sequence that has a level of identity of at least 70% to the nucleotide sequence of the NS1 gene as depicted in SEQ ID NO: 6.

7. (canceled)

8. A non-structural protein 1 (NS1) comprising an amino acid sequence which has a level of identity of at least 70% to the amino acid sequence according to SEQ ID NO: 7.

9. A vaccine for combating RD-associated Erythroparvovirus in equine and/or equine recurrent laryngeal neuropathy, characterised in that said vaccine comprises an immunogenically effective amount of a virus according to claim 1 and a pharmaceutically acceptable carrier.

10. A vaccine according to claim 9, characterised in that said virus is in a live attenuated form.

11. A vaccine according to claim 9, characterised in that said virus is in an inactivated form.

12. A vaccine for combating RD-associated Erythroparvovirus in equine and/or equine recurrent laryngeal neuropathy, characterised in that said vaccine is a subunit vaccine comprising an immunogenically effective amount of a Capsid Protein according to claim 5, and a pharmaceutically acceptable carrier.

13. A vaccine for combating RD-associated Erythroparvovirus in equine and/or equine recurrent laryngeal neuropathy, characterised in that said vaccine is a nucleic acid vaccine comprising a nucleic acid fragment according to claim 3, and a pharmaceutically acceptable carrier.

14. (canceled)

15. A diagnostic test kit for the detection of antibodies reactive with a virus according to claim 1 or the virus itself, characterised in that said test kit comprises a virus according to claim 1, and/or a-Capsid Protein (CP) comprising an amino acid sequence which has a level of identity of at least 70% to the amino acid sequence according to SEQ ID NO: 3 and/or a non-structural protein 1 (NS1) comprising an amino acid sequence which has a level of identity of at least 70% to the amino acid sequence according to SEQ ID NO: 7; wherein the test kit comprises antibodies reactive with the said virus, CP or NS1, or the test kit comprises a PCR primer set that is specifically reactive with a region of the Capsid Protein gene or NS1 gene of the said virus.

16. A vaccine for combating RD-associated Erythroparvovirus in equine and/or equine recurrent laryngeal neuropathy, characterised in that said vaccine is a subunit vaccine comprising an immunogenically effective amount of a non-structural protein 1 according to claim 8, and a pharmaceutically acceptable carrier.

17. The vaccine of claim 16, further comprising an immunogenically effective amount of a capsid protein comprising an amino acid sequence which has a level of identity of at least 70% to the amino acid sequence according to SEQ ID NO: 3.

18. A vaccine for combating RD-associated Erythroparvovirus in equine and/or equine recurrent laryngeal neuropathy, characterised in that said vaccine is a nucleic acid vaccine comprising a nucleic acid fragment according to claim 6, and a pharmaceutically acceptable carrier.

19. A method of combating RD-associated Erythroparvovirus in equine and/or equine recurrent laryngeal neuropathy comprising administering to an equine an immunogenically effective amount of the vaccine of claim 18

20. A method of combating RD-associated Erythroparvovirus in equine and/or equine recurrent laryngeal neuropathy comprising administering to an equine an immunogenically effective amount of the vaccine of claim 17.

21. A method of combating RD-associated Erythroparvovirus in equine and/or equine recurrent laryngeal neuropathy comprising administering to an equine an immunogenically effective amount of the vaccine of claim 13

22. A method of combating RD-associated Erythroparvovirus in equine and/or equine recurrent laryngeal neuropathy comprising administering to an equine an immunogenically effective amount of the vaccine of claim 12.

23. A method of combating RD-associated Erythroparvovirus in equine and/or equine recurrent laryngeal neuropathy comprising administering to an equine an immunogenically effective amount of the vaccine of claim 9, wherein said virus is in a form selected from the group consisting of a live attenuated form and an inactivated form.
